SEQUENCE LISTING (1 sequence)

SEQ ID NO: 1 - protein, 141 aa, Escherichia coli
MAMHLNENLDDNGEMHDINVTPFIDVMLVLLIIFMVAAPLAIVDVKVNLPASTSTPQPRPEKPVYLSVKADNSMFIGNDPVTDETMITALNALTEGKKDITIFFRADKTVDYETLMKVMDTLHQAGYLKIGLVGEETAKAK

(19) United States
(12) Patent Application Publication
(10) Pub. No.: US 2012/0208744 A1
(43) Pub. Date: Aug. 16, 2012

(54) ANTI-MICROBIAL AGENTS AND COMPOSITIONS AND METHODS OF PRODUCTION AND USE THEREOF

(75) Inventor: Kathleen Postle, State College, PA (US)

(73) Assignee: The Penn State Research Foundation, University Park, PA (US)

(21) Appl. No.: 13/371,685

(22) Filed: Feb. 13, 2012

Related U.S. Application Data
(60) Provisional application No. 61/443,511, filed on Feb. 16, 2011.
(60) Provisional application No. 61/532,729, filed on Sep. 9, 2011.

(51) Int. Cl. A61K 38/02; C09D 5/16; A61P 31/04; A01P 1/00; A01N 37/18; A01N 43/713

(52) U.S. Cl. 514/2.8; 106/18.32

(57) ABSTRACT

Anti-microbial compositions for inhibiting gram-negative bacterial growth include inhibitors of one or more interactions between the bacterial proteins TonB and ExbD and/or between the bacterial proteins TolR and TolA. Methods of producing and using the anti-microbial compositions (e.g., treating gram-negative bacterial infections in a mammalian subject) are described. Novel antibiotics that target the TonB system and prevent bacterial growth are described herein.

### CROSS-REFERENCE TO RELATED APPLICATIONS

This application claims the benefit of Provisional Application Ser. No. 61/443,511, filed Feb. 16, 2011, and Provisional Application Ser. No. 61/532,729, filed Sep. 9, 2011, both of which are hereby incorporated by reference in their entirety, for all purposes, herein.

### FEDERALLY SPONSORED RESEARCH

This invention was made with government support under grant no. 2RO1-GM042146 awarded by The National Institutes of Health. The U.S. government has certain rights in the invention.

### FIELD OF THE INVENTION

The invention relates generally to the fields of microbiology, molecular biology, and medicine.

### BACKGROUND

Increasing use of anti-microbials in humans, animals, and agriculture has resulted in many microbes developing resistance to these drugs. Many infectious diseases are increasingly difficult to treat because of antimicrobial-resistant organisms. People infected with antimicrobial-resistant organisms are more likely to have longer hospital stays and may require more complicated treatment. Thus, there is a need for novel anti-microbials that act on new targets and to which microbes will not develop resistance.

### SUMMARY

Described herein are compositions and methods for inhibiting growth of gram-negative bacteria. The compositions and methods include anti-microbial agents that target the TonB and/or Tol systems. In one embodiment, the interaction between the TonB and ExbD proteins (referred to herein as “the TonB/ExbD interaction”) of the TonB system is targeted. To obtain iron, TonB protein must interact with ExbD protein in the periplasm of Escherichia coli and all other Gram negative bacteria where these widespread proteins are present, including all known Gram-negative pathogens except Francisella. By preventing this interaction, the bacteria are prevented from obtaining iron, an essential nutrient, in the iron-limiting environment of a mammalian host. When prevented from obtaining iron, the bacteria cease to grow. At least one region of interaction between TonB and ExbD has been defined. A 30 amino acid region through which TonB and ExbD interact has been carefully defined by the use of cysteine substitutions as described in the Examples section below. In the compositions and methods described herein, residues throughout the entire periplasmic domain of TonB may be targeted. Delivery of inhibitory peptides based on the sequences identified would competitively prevent the TonB-ExbD interaction. If the bacteria mutated to become resistant to the peptide, they would also have become unable to complete the interaction between TonB and ExbD and their growth would thus be blocked. In the methods described herein, anti-microbial agents are delivered to the periplasmic space of the bacteria where the interactions take place. In one embodiment, an anti-microbial agent is a small circular peptide. In another embodiment, an anti-microbial agent is a modified TonB-dependent colicin. TonB-dependent colicins are naturally occurring toxins that cross the outer membrane and gain entry to the periplasmic space by means of the TonB system. Their toxin domains can be exchanged for, or modified to include, peptide sequences of either TonB or ExbD that would prove inhibitory to the normal TonB/ExbD interaction. Thus, described herein are novel antibiotics (anti-microbial agents) that target the TonB system and methods of use thereof. The antimicrobial agents described herein are advantageous, for example, in that should the bacteria become resistant to a particular antimicrobial agent, the bacteria would also become unable to acquire iron by the high-affinity TonB system and thus stop growing for that reason.

The anti-microbial agents (e.g., antibiotics) described herein deliver proteolytically stable peptides to the periplasmic space of bacterial cells. The primary sequence of the peptides is based on any region of interaction between bacterial (e.g., Escherichia coli) ExbD and TonB proteins, and/or between TolR-TolA proteins. Because virtually all Gram-negative bacteria contain the TonB system and pathogens use it to obtain iron, the anti-microbial agents described herein have wide applicability amongst bacteria; species-specific peptides can be developed based on the amino acid sequences and regions of interaction between the various TonB and ExbD proteins (and/or the TolR-TolA proteins). Although the experiments described below pertain to TonB and ExbD protein interactions, there are numerous protein-protein interactions within the TonB system (e.g., TonB/FepA) and/or the Tol system that could be competitively inhibited by anti-microbial agents described herein and stop iron transport or make cells more susceptible to chemotherapeutics.

Unless otherwise defined, all technical terms used herein have the same meaning as commonly understood by one of ordinary skill in the art to which this invention belongs.

As used herein, “protein” and “polypeptide” are used synonymously to mean any peptide-linked chain of amino acids, regardless of length or post-translational modification, e.g., glycosylation or phosphorylation.

By the term “gene” is meant a nucleic acid molecule that codes for a particular protein, or in certain cases, a functional or structural RNA molecule.

As used herein, a “nucleic acid” or a “nucleic acid molecule” means a chain of two or more nucleotides such as RNA (ribonucleic acid) and DNA (deoxyribonucleic acid).

As used herein, “bind,” “binds,” or “interacts with” means that one molecule recognizes and adheres to a particular second molecule in a sample or organism, but does not substantially recognize or adhere to other structurally unrelated molecules in the sample. Generally, when a first molecule “specifically binds” a second molecule, the complex has a dissociation constant (K_{d}) of 10⁻⁴ to 10⁻¹² moles/liter and involves precise “hand-in-a-glove” docking interactions between the two molecules that can be covalent and noncovalent (hydrogen bonding, hydrophobic, ionic, and van der waals).

When referring to a nucleic acid molecule or polypeptide, the term “native” refers to a naturally-occurring (e.g., a wild type, WT) nucleic acid or polypeptide.

“Operably linked” as used herein may mean a functional linkage between two polynucleotides, for example a first polynucleotide and a second polynucleotide, wherein expression of one polynucleotide affects transcription and/or translation and/or mRNA stability of the other polynucleotide. “Operably linked” may also mean a functional linkage between two or more polypeptides (e.g., a first polypeptide and a second polypeptide).

A “high throughput screen” or “HTS” as used herein refers to an assay which provides for multiple candidate agents, samples or test compounds to be screened simultaneously. As further described below, examples of such assays may include the use of microtiter plates that are especially convenient because a large number of assays can be carried out simultaneously, using small amounts of reagents and samples. The methods are easily carried out in a multi-well format including, but not limited to, 96-well and 384-well formats and automated.

As used herein, the term “anti-microbial agent” is meant to encompass any molecule, chemical entity, composition, drug, therapeutic agent, or biological agent capable of preventing or reducing growth of a microbe, or capable of blocking the ability of a microbe to cause disease. An example of an anti-microbial agent is an antibiotic. The term includes small molecule compounds, antisense reagents, siRNA reagents, antibodies, enzymes, peptides, organic or inorganic molecules, natural or synthetic compounds and the like.

The terms “patient,” “subject” and “individual” are used interchangeably herein, and mean a mammalian (e.g., human) subject to be treated and/or to obtain a biological sample from.

As used herein, the term “treatment” is defined as the application or administration of a therapeutic agent to a patient or subject, or application or administration of the therapeutic agent to an isolated tissue or cell line from a patient or subject, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a composition as described herein effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

Accordingly, described herein is a pharmaceutical composition including an inhibitor (e.g., a peptide) of a TonB/ExbD interaction in a therapeutically effective amount for inhibiting growth of gram-negative bacteria in a subject and a pharmaceutically acceptable carrier. In one embodiment, the peptide is a cyclic peptide of approximately 9 or more amino acid residues in length and is produced by Split Intein Circular Ligation of Proteins and Peptides (SICLOPPS) methodology. The cyclic peptide inhibitor can be an antibiotic. The inhibitor can be a modified colicin protein, e.g., a modified colicin protein that is an antibiotic. The gram-negative bacteria can be any gram-negative bacteria. A non-exhaustive list of gram-negative bacteria includes: Escherichia coli, Salmonella enterica serovar typhi, Vibrio cholerae, Burkholderia pseudomallei, Klebsiella pneumoniae, Pseudomonas aeruginosa, Acinetobacter baumanii, and Yersinia enterocolitica. In a typical embodiment, the inhibitor reduces uptake of iron by the gram-negative bacteria. The inhibitor may bind to a region of ExbD including amino acids 42-61, 62-141, 92-121, or other specific regions as identified herein, or to a region of TonB including amino acids 33-239 of TonB or other specific regions as identified herein.

Also described herein is a method of inhibiting growth of gram-negative bacteria in a subject (e.g., human). The method includes the steps of: providing a pharmaceutical composition including an inhibitor of a TonB/ExbD interaction in a therapeutically effective amount for inhibiting growth of gram-negative bacteria in the subject and a pharmaceutically acceptable carrier; and administering the pharmaceutical composition to the subject. In the method, the inhibitor typically enters the periplasmic space of the gram-negative bacteria and inhibits their growth, and is typically a peptide, e.g., a 9-mer or larger cyclic peptide produced by SICLOPPS methodology. A cyclic peptide inhibitor can be an antibiotic. The inhibitor can be a modified colicin protein (e.g., a modified colicin protein that is an antibiotic). The gram-negative bacteria can be any gram-negative bacteria (e.g., any one of those listed herein). The inhibitor may decrease uptake of iron by the gram-negative bacteria. The inhibitor may bind to a region of ExbD including amino acids 42-61, 62-141, 92-121, or other specific regions as identified herein, or to a region of TonB including amino acids 33-239 of TonB, or other specific regions as identified herein.

Additionally described herein is a method of inhibiting growth of gram-negative bacteria on a solid surface (e.g., at least one surface of a medical device or any other solid surface subject to biofouling). Generally, the method includes: providing a composition including an inhibitor (e.g., a peptide) of a TonB/ExbD interaction in a therapeutically effective amount for inhibiting growth of gram-negative bacteria; and coating the solid surface with the composition in an amount effective for inhibiting growth of the gram-negative bacteria on the solid surface. In the method, the inhibitor enters the periplasmic space of the gram-negative bacteria and inhibits their growth. The inhibitor can be a peptide that is approximately a 9-mer or larger cyclic peptide produced by SICLOPPS methodology (e.g., a cyclic peptide inhibitor that is an antibiotic). The inhibitor can be a modified colicin protein (e.g., a modified colicin protein that is an antibiotic). The gram-negative bacteria can be any gram-negative bacteria (e.g., any one of those listed herein). The inhibitor typically reduces uptake of iron by the gram-negative bacteria. The inhibitor may bind to a region of ExbD including amino acids 42-61, 62-141, or 92-121 of ExbD, or other specific regions as identified herein, or to a region of TonB including amino acids 33-239 of TonB or other specific regions as identified herein.

Still further described herein is a pharmaceutical composition including an inhibitor (e.g., a peptide) of a TolR-TolA interaction in a therapeutically effective amount for inhibiting growth of gram-negative bacteria in a subject and a pharmaceutically acceptable carrier. In the composition, the inhibitor may be a peptide that is approximately a 9-mer or larger cyclic peptide produced by SICLOPPS methodology (e.g., a cyclic peptide inhibitor that is an antibiotic). The inhibitor can be a modified colicin protein (e.g., a modified colicin protein that is an antibiotic).

Yet further described herein is a method of inhibiting growth of gram-negative bacteria in a subject (e.g., human). Generally, the method includes the steps of: providing a pharmaceutical composition including an inhibitor (e.g., a peptide) of a TolR-TolA interaction in a therapeutically effective amount for inhibiting growth and/or decreasing the high native resistance to antibiotics, by disrupting the outer membranes of the gram negative bacteria in the subject and a pharmaceutically acceptable carrier; and administering the pharmaceutical composition to the subject. The inhibitor enters the periplasmic space of the gram-negative bacteria and inhibits their growth. In one embodiment, the inhibitor is a peptide that is approximately a 9-mer or larger cyclic peptide produced by SICLOPPS methodology (e.g., a cyclic peptide inhibitor that is an antibiotic. The inhibitor can be a modified colicin protein (e.g., a modified colicin protein that is an antibiotic).

A method of inhibiting growth of gram-negative bacteria on a solid surface (e.g., at least one surface of a medical device or any other solid surface susceptible to biofouling) is also described herein. Generally the method includes: providing a composition including an inhibitor (e.g., a peptide) of a TolR-TolA or a TonB-ExbD interaction in a therapeutically effective amount for inhibiting growth of gram-negative bacteria and/or disrupting the outer membranes of the gram negative bacteria and decreasing the high native resistance to antibiotics, and coating the solid surface with the composition in an amount effective for inhibiting growth of the gram-negative bacteria on the solid surface. The inhibitor enters the periplasmic space of the gram-negative bacteria and inhibits their growth. The inhibitor can be a peptide that is approximately a 9-mer or larger cyclic peptide produced by SICLOPPS methodology (a cyclic peptide inhibitor that is an antibiotic). The inhibitor can be a modified colicin protein (e.g., a modified colicin protein that is an antibiotic).

Although compositions and methods similar or equivalent to those described herein can be used in the practice or testing of the present invention, suitable compositions and methods are described below. All publications, patent applications, and patents mentioned herein are incorporated by reference in their entirety. In the case of conflict, the present specification, including definitions, will control. The particular embodiments discussed below are illustrative only and not intended to be limiting.

### BRIEF DESCRIPTION OF THE DRAWINGS

FIG. 1 is a model for initial stages in TonB energization. Three sequential stages in TonB energization in the cytoplasmic membrane (CM) are shown from left to right. ExbB, assumed to be present for all stages, is not shown. Black constructs with filled transmembrane domains represent TonB; gray constructs with empty transmembrane domains represent ExbD. Jagged regions represent disordered domains. This model is not drawn to scale and represents a conceptual framework only. It depicts that the interaction of TonB and ExbD through their periplasmic domains is essential for subsequent TonB activity in transporting iron across the outer membrane (Stage 1V, not shown).

FIG. 2 shows consecutive 10 residue deletions span the ExbD protein. A, The E. coli ExbD amino acid sequence is shown (SEQ ID NO:1), with consecutive deleted regions indicated above. The predicted secondary structure of ExbD is depicted as arrows for β-sheets and cylinders for α-helices. Secondary structure prediction used the PredictProtein Server, PROFsec output. The predicted topological location of these amino acids is depicted to the right. The topology depiction is not intended to reflect predicted tertiary structure. B, Periplasmic domain deletions are mapped on the ExbD NMR structure (pdb code: 2 pfu). Consecutive deletions alternate between black and grayed ribbon for distinction. The initiating reside of each deletion is labeled at the relative start of the deletion, with the exception of Δ42-51 since the structure starts at residue 43. To facilitate reading of the labels, positioning may not mark the exact residue location on the ribbon. The image was generated using Swiss-PdbViewer.

FIG. 3 shows that interactive cys substitutions do not form one interface on the ExbD NMR structure. Side chains of residues where cys substitutions were trapped in disulfide-linked homodimers in vivo are mapped on the ExbD periplasmic domain NMR structure, pdb code 2 pfu. Grayed ribbon indicates the cys scanned region examined in this study. Black side chains indicate significant spontaneous ExbD homodimer formation. Gray side chains indicate weak spontaneous homodimer formation. All side chains pictured showed significant ExbD heterodimer formation with TonB A150C formation “C” and “N” indicate the carboxy and amino terminus of the domain, respectively.

FIG. 4 shows mapping of specific sites of TonB-ExbD periplasmic domain interactions supported by a functional ExbD TMD. ExbD and TonB cysteine substitutions examined and their abilities to form disulfide crosslinked heterodimers are depicted. TonB cys substitutions are listed on the left and right, and the ExbD cys substitution each was co-expressed with is listed in the center. Solid lines indicate a relatively strong interaction. TonB substitutions supporting strong interactions are highlighted in bold. Dashed lines indicate a relatively weak or inefficient interaction. A-C, Interactions supported by ExbD cys substitutions with a wild-type ExbD TMD.

FIG. 5 shows that strong and weak interactions are supported by ExbD A92C and TonB substitutions between N200C and R214C. A strain expressing wild-type ExbD and TonB (W3110) or a ΔexbD, ΔtonB, ΔtolQR strain (KP1509) co-expressing ExbD A92C (pKP1000) with TonB C18G, N200C (pKP469), F202C (pKP415), R204C (pKP418), V206C (pKP463), N208C (pKP416), M210C (pKP466), R212C (pKP471), or R214C (pKP473) were processed in non-reducing sample buffer containing iodoacetamide as described in Materials and Methods of Example 4. Samples were resolved on a 13% non-reducing SDS-polyacrylamide gel and immunoblotted with ExbD-specific polyclonal antibodies (left) or TonB-specific monoclonal antibodies (right). Samples from the same cultures were processed in reducing sample buffer containing βME and resolved on 11% or 13% SDS-polyacrylamide gels and immunoblotted with TonB-specific monoclonal or ExbD-specific polyclonal antibodies (lower immunoblots). The combination of substitutions specific to each lane are indicated across the top. Positions of molecular mass standards are indicated in the center. Positions of the ExbD or TonB monomers and disulfide-linked complexes are indicated on the left and right, respectively.

FIG. 6 shows ExbD cys substitutions showing strong interactions with TonB cys substitutions in vivo map to opposite ends of the ExbD periplasmic domain NMR structure. The locations of the six ExbD cys substitutions examined in this study, with side chains of the native residue shown, are mapped on the ExbD periplasmic domain NMR structure, pdb code 2 pfu. The image was generated using Swiss-PdbViewer. Black side chains indicate sites of significant spontaneous ExbD-TonB heterodimer formation. Gray side chains indicate sites of no significant spontaneous heterodimer formation. “C” and “N” indicate the carboxy and amino terminus of the domain, respectively.

### DETAILED DESCRIPTION

Described herein are anti-microbial compositions for inhibiting gram-negative bacterial growth, and methods of producing and using thereof. Experiments that further an understanding of the mechanism of signal transduction of the TonB system at the molecular level are described herein. Based on these experimental results, novel antibiotics that target the TonB system and/or homologous Tol system and prevent bacterial growth are described herein. The TonB system is involved in iron transport, while the Tol system is involved in outer membrane integrity. Also in some bacteria, the Tol system appears to be essential for cell division. The TonB and Tol systems are unique targets in that, if they are functioning, they may be susceptible to inhibition by the antibiotics described herein. Advantageously, if a component of the TonB system mutates to develop antibiotic resistance, the cells will be disabled by an inability to obtain iron and their growth will be blocked. If a component of the Tol system mutates to develop antibiotic resistance, the cells will be disabled by an inability to maintain an intact outer membrane barrier and thus remain highly susceptible to the antibiotic.

### The TonB System in Gram-Negative Bacteria

Gram-negative bacteria have two concentric membranes, inner and outer. The inner membrane is similar to the plasma membranes of all living organisms and contains the enzymes that generate and maintain a proton electrochemical gradient (called proton motive force, or pmf). It also contains the primary and secondary active transporters that bring most nutrients into the cells. The outer membrane is punctuated with proteinaceous pores that allow most nutrients free diffusion access to the periplasmic space between the two membranes; unlike the inner membrane, it is not energized by ion gradients or access to ATP. From that point all nutrients can be actively transported across the inner membrane. Thus the outer membrane constitutes a barrier to large, scarce, important nutrient acquisition, for example the acquisition of iron-siderophores. Iron forms the basis for a tug of war between pathogen and host. Mammalian hosts maintain low free serum iron concentrations by use of iron-binding proteins such as lactoferrin and transferrin.

The innate immune response to systemic bacterial infection is to attempt to lower serum iron levels still further: the intestines stop taking it up and the liver starts to sequester it. Many pathogenic Gram-negative bacteria use creative approaches to counter host efforts to prevent iron acquisition. One such approach is the TonB system, which in Neisseria sp., for example, can snatch an iron atom out of host transferrin. Described below are experiments that investigate the TonB system of Gram-negative bacteria, using non-pathogenic Escherichia coli K12 as a model system. The TonB system overcomes the outer membrane bather by using the pmf of the cytoplasmic membrane. A complex of proteins in the cytoplasmic membrane TonB/ExbB/ExbD harvests the pmf and transmits the energization signal to high-affinity transport proteins (such as FepA) in the outer membrane. Adding to its importance, the TonB system is used wherever Gram-negative bacteria require energization of outer membrane transport proteins. TonB-dependent transporters such as FepA are widely present (Xanthomonas has 48), with each specific for a transport ligand that can range from iron-chelator complexes (Fe-siderophores) to Ni++ to maltodextrins.

Although the experiments described herein pertain primarily to the TonB/ExbD interaction in gram-negative bacteria, any other suitable protein-protein interactions in the TonB system can be targeted by the anti-microbial compositions described herein. Suitable protein-protein interactions include those, for example, required for iron transport into gram-negative bacterial cells. For example, the physical interaction of TonB with any of the TonB-dependent outer membrane transporters such as FepA in E. coli or TbpA in Neisseria species.

### Biological Methods

Methods involving conventional molecular biology techniques are described herein. Such techniques are generally known in the art and are described in detail in methodology treatises such as Molecular Cloning: A Laboratory Manual, 3rd ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 1992 (with periodic updates). Construction and use of a SICLOPPS (Split Intein Circular Ligation of Proteins and Peptides) library is described in Scott et al., Proc. Natl. Acad. Sci. vol. 96:13638-13643, 1999; Tavassoli et al., Nature Prot. Vol. 12:1126-1133, 2007; and U.S. Pat. Nos. 7,354,756 and 7,846,710. Immunology techniques are generally known in the art and are described in detail in methodology treatises such as Advances in Immunology, volume 93, ed. Frederick W. Alt, Academic Press, Burlington, Mass., 2007; Making and Using Antibodies: A Practical Handbook, eds. Gary C. Howard and Matthew R. Kaser, CRC Press, Boca Raton, Fl, 2006; Medical Immunology, 6^{th} ed., edited by Gabriel Virella, Informa Healthcare Press, London, England, 2007; and Harlow and Lane ANTIBODIES: A Laboratory Manual, Cold Spring Harbor Laboratory Press, Cold Spring Harbor, N.Y., 1988. The nucleic acid sequences encoding the proteins of the TonB system, as well as the amino acid sequences of the proteins, are known. For example, the amino acid sequence of E. coli TonB is BAA14784.2, and the amino acid sequence of E. coli ExbD is BAE77063.1

### Pharmaceutical Compositions for Inhibiting Growth of Gram-Negative Bacteria

Pharmaceutical compositions for inhibiting growth of gram-negative bacteria are described herein. A typical pharmaceutical composition includes an inhibitor of a TonB/ExbD interaction (an antibiotic) in a therapeutically effective amount for inhibiting growth of gram-negative bacteria in a subject and a pharmaceutically acceptable carrier. Such an inhibitor can reduce virulence or inhibit growth of a microbe. Generally, the inhibitor is a peptide. For example, 9-mer or larger cyclic peptides produced by SICLOPPS methodology are described below (see, e.g., Cheng et al., Protein Sci. 2007 August; 16(8):1535-42). The cyclic peptides can cross the outer membranes of numerous gram negative bacteria by diffusion through porins. For those gram negative bacteria such as Pseudomonas aeruginosa or Acinetobacter baumanii where their high intrinsic resistance is partly due to low permeability of the outer membrane, an alternative is to couple the inhibitory peptides to bacteriocins, ribosomally encoded antimicrobial peptides (e.g. colicins for E. coli, pesticins for Y. pestis, pyocins for P. aeruginosa). In the case of E. coli, the inhibitor is a modified colicin protein whose toxic domain has been replaced with a large stretch of amino acids that inhibits at least one protein-protein interaction in the TonB system (e.g., TonB/ExbD). For colicin B, the structure is known (1RH1). The receptor/translocation domain that recognizes FepA and allows translocation across the outer membrane is well separated from the toxin domain. It is the toxin domain that is to be replaced by inhibitory peptide sequences. The advantage of this approach is that larger regions of the ExbD or TonB domains can be delivered to the periplasm. As an example, the TonB region from 150 to 239 could be substituted for the colicin B toxin domain. This would then serve as a competing protein to interrupt the interaction between ExbD and TonB or TonB and all of the TonB-dependent transporters. The growth of any gram-negative bacteria may be prevented or inhibited using the pharmaceutical compositions described herein. Examples of gram-negative bacteria include but are not limited to Escherichia coli, Salmonella enterica serovar typhi, Vibrio cholerae, Burkholderia pseudomallei, Klebsiella pneumoniae, Pseudomonas aeruginosa, Acinetobacter baumanii, and Yersinia enterocolitica. For those gram negative bacteria not sensitive to colicins, bacteriocins for such gram negative bacteria are known Inhibitors of at least one protein-protein interaction in the TonB system (e.g., TonB/ExbD) modulate uptake of iron by the gram-negative bacteria. As an example, the ability to block ExPEC E. coli (extraintestinal pathogenic E. coli) from acquiring iron through TonB-dependent hemoreceptors ChuA and Hma, or through siderophore acquisition systems would be used to limit ExPEC-related processes in livestock [Fairbrother and Nadeau (2010) Colibacillosis In Doc, E.T. (Ed.) Infections and Parasitic Diseases of Livestock, Lavoisier, Paris pp 917-945].

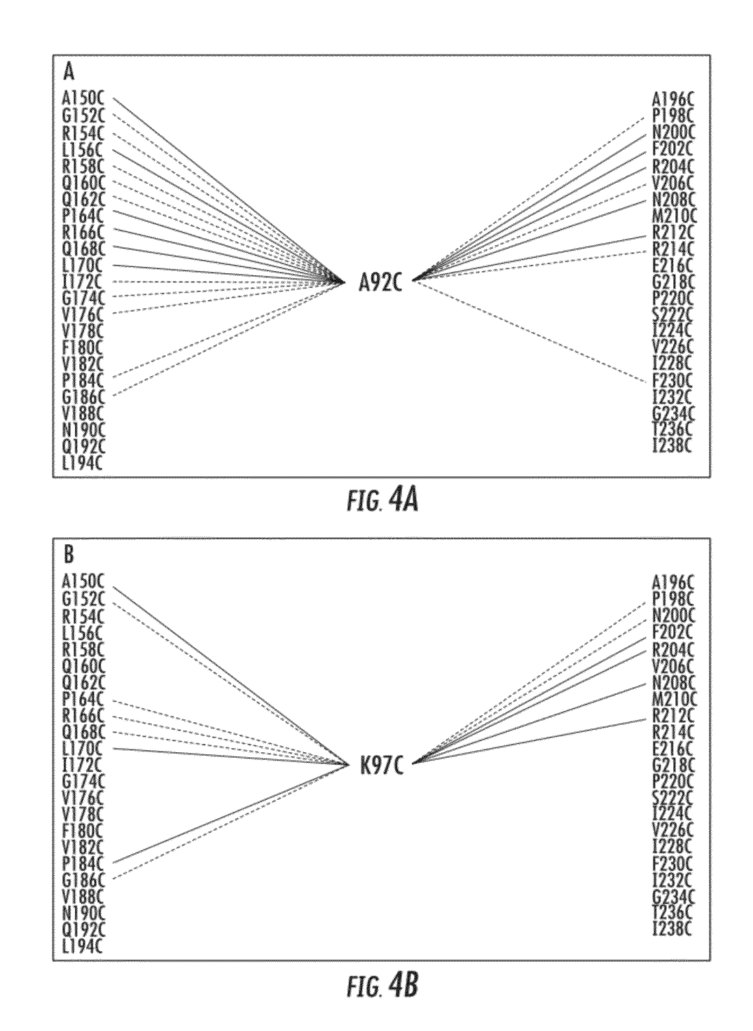

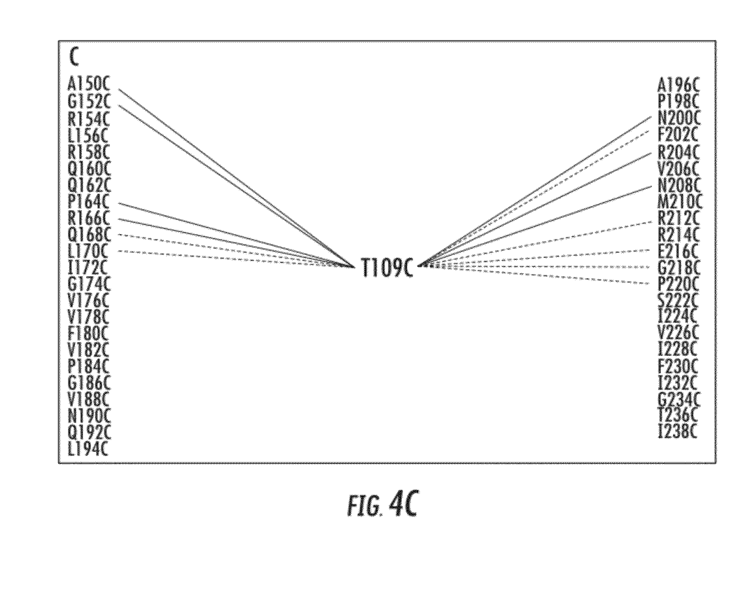

In Example 1 below, experiments that elucidated the particular amino acids of each of TonB and ExbD that are required for their interaction (and for uptake of iron by gram-negative bacterial cells) were performed. The entire periplasmic domain of either TonB or ExbD could be used as an inhibitor. As an alternative to a colicin-type system, various interacting regions of ExbD and TonB have been mapped directly for use in the circular peptides and the SICLOPPS system. Inteins are naturally occurring protein splicing sequences that can be used to ligate N and C termini of a peptide to circularize it. Basically, the desired codon sequence is engineered between C-terminal (I_{C}) and N-terminal (I_{N}) intein coding sequences. Recovery of cyclic peptides for characterization and purification is aided by a chitin binding domain (CBD) fused to the C-terminus of I_{N}. Expression of the plasmid encoded fused I_{C}-ExbD codons-I_{N}-CBD peptide is induced, cells are lysed by French press in buffer appropriate for the chitin column and the lysate is allowed to process on the chitin column down to the circularized peptide and various intermediates. The particular amino acids of ExbD that directly interact with TonB in vivo can be localized to a region from 92-121. Within that region it is known that ExbD aa92, 97, and 109 make direct contact with TonB 150, 152, and a region from 200-212 (see FIG. 4). Circular peptide inhibitors would be based on short peptide sequences centered on residue 92, for example. Alternatively, the sequences from 200-212 of TonB could be used. Additional candidates are ExbD amino acids 42-61 of ExbD that are required for pmf-dependent conformational changes in the ExbD periplasmic domain to allow homodimerization. There may also be other regions to target, since the ExbD L132Q substitution is inactive and prevents formation of the TonB-ExbD formaldehyde crosslink. Alternative targets of TonB interaction with FepA have been identified. The region from 200-212 is also seen to be important for interactions with TonB-dependent transporters (Ghosh, J & Postle, K, Mol. Microbiol. 2005 January; 55(1):276-88; Postle et al., MBio. 2010 Dec. 21; 1(5). pii: e00307-10). Applicant has determined that TonB F202 and W213 interact with FepA D422 in one of the periplasmic loops in vivo. Given the overall structural homology among transporters (22-stranded beta-barrels with N-terminal internal globular domains that occlude the lumen) a wide variety of inhibitory peptides can be developed, targeting the TonB system of the pathogen of interest. Finally, the interaction of TonB with the TonB box of the various transporters would be a target for inhibition by a circular peptide. The TonB box is already a known region of in vivo interaction between TonB and its cognate transporters, e.g. BtuB Site-directed disulfide bonding reveals an interaction site between energy-coupling protein TonB and BtuB, the outer membrane cobalamin transporter (Cadieux N, Kadner R J. (1999) Proc Natl Acad Sci USA. 96(19):10673-8).

The pharmaceutical compositions can be administered to any mammalian subject. Examples of mammalian subjects include humans, non-human primates, bovines, canines, equines, etc. The amount of the pharmaceutical composition (therapeutic agent) to be administered varies depending upon the manner of administration, the age and body weight of the patient, and with the pathology of the disease. A composition as described herein is typically administered at a dosage that inhibits growth of pathogenic gram-negative bacteria in the subject. The therapeutic methods of the invention (which include prophylactic treatment), in general, include administration of a therapeutically effective amount of a composition described herein to a subject (e.g., animal, human) in need thereof, including a mammal, particularly a human. Such treatment will be suitably administered to subjects, particularly humans, suffering from, having, susceptible to, or at risk for a disease associated with and/or caused by gram-negative bacteria, or symptom thereof.

Pharmaceutical compositions described herein can be administered to a subject by any suitable delivery vehicle and route. The administration of a pharmaceutical composition including a therapeutically effective amount of an inhibitor of a TonB-ExbD interaction or TolR-TolA interaction may be by any suitable means that results in a concentration of the inhibitor of the TonB-ExbD or TolR-TolA interaction that is effective in inhibiting growth of gram-negative bacteria in a subject. An inhibitor of a TonB-ExbD or TolR-TolA interaction as described herein may be contained in any appropriate amount in any suitable carrier substance, and is generally present in an amount of 1-95% by weight of the total weight of the composition. The composition may be provided in a dosage form that is suitable for local or systemic administration (e.g., parenterally, subcutaneously, intravenously, intramuscularly, intraperitoneally). The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

### Methods of Producing and Delivering Anti-Microbial Agents

Any suitable means for producing stable peptides for use as anti-microbials and delivering such peptides to the periplasmic space of gram-negative bacteria may be used. For example, a modified protein such as a modified bacteriocin protein could be generated. Numerous toxin proteins have evolved to use outer membrane proteins to cross the outer membrane. For E. coli, these are called colicins. These are large proteins in the 50-60 kDa range. The domains important for translocation across the outer membrane through the proteins similar to FepA would remain intact. The toxic domains of the colicins would be replaced with amino acids sequences that would inhibit the TonB-ExbD or TolA-TolR interaction to create the colicin delivery system. Modified colicin peptides would be engineered into plasmid clones of colicin genes under the control of strong inducible promoters such as the lac or phage T7 promoters. The engineered system would also include in frame His 10 or glutathione-S-transferase (GST) tags that may or may not be cleavable. Bacterial cells containing high levels of a colicin delivery peptide would be lysed and the resultant peptides purified on the appropriate affinity column matrix. In this embodiment, peptides are delivered using the TonB system itself and would inhibit either the TonB system or the Tol system. It is not a requirement that the bacteriocin delivery peptides use a specific system to cross the outer membrane—only that they can do so.

As another example, circular peptides that are smaller in size for transit across the outer membrane through the porin proteins may be engineered using methods of producing cyclic peptides and splicing intermediates of peptides in a looped conformation, known as SICLOPPS (Split Intein Circular Ligation of Proteins and Peptides) are disclosed, for example, in Scott et al., Proc. Natl. Acad. Sci. vol. 96:13638-13643, 1999; Tavassoli et al., Nature Prot. Vol. 12:1126-1133, 2007; and U.S. Pat. Nos. 7,354,756 and 7,846,710. As described in these references, the methods utilize the trans-splicing ability of split inteins to catalyze cyclization of peptides from a precursor peptide having a target peptide interposed between two portions of a split intein. The interaction of the two portions of the split intein creates a catalytically-active intein and also forces the target peptide into a loop configuration that stabilizes the ester isomer of the amino acid at the junction between one of the intein portions and the target peptide. A heteroatom from the other intein portion then reacts with the ester to form a cyclic ester intermediate. The active intein catalyzes the formation of an aminosuccinimide that liberates a cyclized form of the target peptide, which spontaneously rearranges to form the thermodynamically favored backbone cyclic peptide product. An advantage of producing peptides for inhibiting gram-negative bacterial growth by SICLOPPS methodology is that the resultant peptides are resistant to being proteolytically degraded in the periplasmic space of the bacterial cells. Typically, the targets of the cyclic peptides are in the periplasmic space so all they have to do is cross the outer membrane. The SICLOPPS methodology can be used to produce any circular peptides that compete with any periplasmically localized TonB interaction that will result in blocking uptake of iron by gram-negative bacteria cells (and thus prevention of growth of the bacterial cells) Likewise, blockage of any Tol interaction would render the outer membranes of the cells more permeable to antibiotics or to circular peptides of greater size. For example, SICLOPPS-produced circular peptides that target any region of either TonB or FepA that is involved in the TonB/FepA interaction can be used to inhibit gram-negative bacterial growth (see Devanathan and Postle, 2007 Mol. Microbiol. 2007 65:441-53; Studies on colicin B translocation: FepA is gated by TonB). As another example, SICLOPPS-produced circular peptides that target any region of ExbD that is required for iron uptake can be used to inhibit gram-negative bacterial growth.

Unlike some SICLOPPS-based methods of screening libraries, however, production of anti-microbial agents as described herein would not require screening libraries of peptides. They would instead be rationally designed based on information described herein about interacting regions between ExbD and TonB.

One or more systems, methods or both can be used to identify a candidate anti-microbial agent (e.g., antibiotic). Manual systems/methods, semi-automated systems/methods, and automated systems/methods are all possible. A variety of robotic or automatic systems are available for automatically or programmably providing predetermined motions for handling, contacting, dispensing, or otherwise manipulating materials in solid, fluid liquid or gas form according to a predetermined protocol. Such systems may be adapted or augmented to include a variety of hardware, software or both to assist the systems in determining mechanical properties of materials. Hardware and software for augmenting the robotic systems may include, but are not limited to, sensors, transducers, data acquisition and manipulation hardware, data acquisition and manipulation software and the like. Exemplary robotic systems are commercially available from CAVRO Scientific Instruments (e.g., Model NO. RSP9652) or BioDot (Microdrop Model 3000).

After a candidate anti-microbial agent is identified, pharmacokinetic profiling can by pursued by administration of an amount of the agent to an animal host as a suitable animal model. After further efficacy and safety analysis, the drug can be administered to a human patient to treat or ameliorate the symptoms of disease caused by a microbial infection (e.g., bacterial infection).

### Methods of Inhibiting Growth of Gram-Negative Bacteria in a Subject

A method of inhibiting growth of gram-negative bacteria in a subject typically includes the steps of: providing a pharmaceutical composition including an inhibitor of a TonB/ExbD interaction (an antibiotic) in a therapeutically effective amount for inhibiting growth of gram-negative bacteria in the subject and a pharmaceutically acceptable carrier; and administering the pharmaceutical composition to the subject. The subject is typically mammalian, e.g., a human. In the methods, the inhibitor enters the periplasmic space of the gram-negative bacteria and inhibits their growth. In one embodiment, the inhibitor is a peptide, e.g., a 9-mer cyclic peptide that is produced by SICLOPPS methodology described above. In another embodiment, the inhibitor is a modified bacteriocin protein. The methods can be used to treat a subject infected by any gram-negative bacteria, e.g., Escherichia coli, Salmonella enterica serovar typhi, Vibrio cholerae, Burkholderia pseudomallei, Klebsiella pneumoniae, Pseudomonas aeruginosa, Acinetobacter baumanii, and Yersinia enterocolitica. In the methods, the inhibitor prevents growth of the gram-negative bacteria by modulating (inhibiting) uptake of iron by the gram-negative bacteria. In the methods, the inhibitor can bind to any suitable region of a TonB system protein that interferes with binding of that protein to another TonB system protein, resulting in inhibition of iron uptake by the bacteria. As described above, the particular amino acids of ExbD that directly interact with TonB in vivo can be localized to a region from 92-121 (e.g., it is known that ExbD aa 92, 97, and 109 make direct contact with TonB 150, 152, and a region from 200-212; e.g., circular peptide inhibitors would be based on short peptide sequences centered on residue 92). Also as described above, the sequences from 200-212 of TonB could be used, and additional candidates are ExbD amino acids 42-61 of ExbD that are required for pmf-dependent conformational changes in the ExbD periplasmic domain to allow homodimerization. Although the experiments described in Example 1 below primarily pertain to inhibiting growth of E. coli, the compositions and methods described herein can be used to inhibit the growth of other microbes such as Klebsiella pneumoniae (Hsieh et al., J Infect Dis. 2008 Jun. 15; 197(12):1717-27). To design an anti-microbial as described herein for inhibiting the growth of a particular microbe other than E. coli, regions of homology between the known interacting regions of E. coli TonB and ExbD or outer membrane transporters would be used to design circular peptide sequences and inhibitiory bacteriocin delivery systems. This would work for any gram negative bacterium with sufficient homology or where reasonable guesses about same could be made. It would also work for any gram negative bacterium for which bacteriocin genes could be identified.

### Additional Uses for the Inhibitors Described Herein

The anti-microbial compositions described herein can be used for applications other than as pharmaceuticals. Potential applications include incorporation in or surface coating of materials for medical use such as stents or artificial valves or in non-medical devices, inclusion in other formulations to provide microbiocidal activity such as additives for cleaning solutions, water treatment, and general prevention of biofouling. For example, a composition including an inhibitor of a TonB/ExbD interaction or a TolR-TolA interaction produced by the methods described herein can be used to inhibit bacterial growth on a solid surface. A solid surface (e.g., at least one surface of a medical device) can be coated with a composition including an inhibitor of a TonB/ExbD and/or TolR-TolA interaction in an amount effective for inhibiting bacterial growth on the solid surface.

### Data and Analysis

Use of the methods and compositions described herein may employ conventional biology methods, software and systems. Useful computer software products typically include computer readable medium having computer-executable instructions for performing logic steps of a method. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are encompassed by the methods described herein, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The methods described herein may also make use of various computer program products and software for a variety of purposes, such as reagent design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170. Additionally, the embodiments described herein include methods for providing data (e.g., experimental results, analyses) and other types of information over networks such as the Internet.

### Compositions and Methods for Inhibiting Growth of Gram-Negative Bacteria in a Subject Involving Targeting of the Tol System

As with the TonB system, a pharmaceutical composition for inhibiting growth of gram-negative bacteria in a subject can include an inhibitor of a TolR-TolA interaction (an antibiotic). The Tol system is sufficiently similar to the TonB system that there is functional crosstalk between them (Eick-Helmerich and Braun, J. Bacteriol. 1989 September; 171(9):5117-26; Braun and Hermann, Mol. Microbiol. 1993 April; 8(2):261-8). ExbD and TolR have similarity in their sequences. The Tol system is involved in maintenance of OM integrity (Cascales et al., Microbiol Mol Biol Rev. 2007 March; 71(1):158-229). Using the methods described herein for targeting the TonB system, the Tol system can be targeted by a cyclic peptide, proving an important adjunct to many different antibiotics, including those targeting the TonB system, by making the OM leaky. Typically, the regions of TolR most likely to interact with TolA would be used as templates for engineered circular peptides. The test of the peptides is whether they can decrease the minimal inhibitory concentration of antibiotics, such as vancomycin, which are commonly used to assay OM leakiness induced in Tol system mutants. Upon identifying such peptides, larger (and thus potentially more potent) ExbD-specific circular peptides may be engineered.

In one embodiment of a pharmaceutical composition, the composition includes an inhibitor of a TolR-TolA interaction in a therapeutically effective amount for inhibiting growth of gram-negative bacteria in a subject and a pharmaceutically acceptable carrier. In one embodiment of a method of inhibiting growth of gram-negative bacteria in a subject (e.g., human), the method includes the steps of: providing a pharmaceutical composition including an inhibitor of a TolR-TolA interaction in a therapeutically effective amount for inhibiting growth and/or decreasing the high native resistance due to limited permeability of the outer membranes of gram-negative bacteria in the subject and a pharmaceutically acceptable carrier; and administering the pharmaceutical composition to the subject. In one embodiment of a method of inhibiting growth of gram-negative bacteria on a solid surface (e.g., at least one surface of a medical device or any other solid surface susceptible to biofouling), the method includes providing a composition including an inhibitor of a TolR-TolA interaction in a therapeutically effective amount for inhibiting growth of gram-negative bacteria and/or decreasing the high native resistance due to limited permeability of the outer membranes of gram-negative bacteria; and coating the solid surface with the composition in an amount effective for inhibiting growth of the gram-negative bacteria on the solid surface.

In all of the above embodiments, the inhibitor can be a peptide such as a 9-mer or larger cyclic peptide produced by SICLOPPS methodology. The cyclic peptide inhibitor can be an antibiotic. The inhibitor can be a modified colicin protein (e.g., an antibiotic). Generally in these embodiments, the inhibitor enters the periplasmic space of the gram-negative bacteria and inhibits their growth.

### EXAMPLES

The present invention is further illustrated by the following specific examples. The examples are provided for illustration only and should not be construed as limiting the scope of the invention in any way.

### Example 1

### ExbD Mutants Define Initial Stages in TonB Energization

This study provided the first evidence for pmf-dependent conformational changes in ExbD, with pmf acting as a toggle switch for both ExbD and TonB conformations in spheroplasts. The ExbD-TonB formaldehyde crosslink also formed in spheroplasts, indicating that they provided a biologically relevant system for study of in vivo conformational changes. Asp25 in the ExbD transmembrane domain, currently the best candidate to be on a proton translocation pathway, was essential for both TonB and ExbD conformational response to pmf. Three stages in TonB energization were defined. The first stage represented lack of interaction between the TonB and ExbD periplasmic domains. Second was an initial assembly of the TonB and ExbD periplasmic domains that rendered each resistant to proteinase K. ExbD D25N left the TonB conformation stalled at stage two, as did collapse of the pmf. The third corresponded with the previously identified pmf-dependent, energized interaction between TonB and ExbD. These observations supported a model where ExbD couples TonB to the pmf, with concomitant transitions of ExbD and TonB periplasmic domains from unenergized to energized heterodimers.

These results demonstrated that ExbD and TonB are toggled between two different conformations with pmf as the switch. The ExbD TMD (Asp25) was required to mediate this conformational switch. The ExbD carboxy-terminus (Leu132) was important for staging the initial ExbD-TonB interaction. This also constituted the first evidence that ExbD conformation was pmf-dependent.

### Results

Loss of protonmotive force reversibly stalls TonB conformational changes. Previous work has shown the conformation of the TonB carboxy terminus to be pmf-responsive. In spheroplasts, TonB is completely sensitive to exogenous proteinase K, as expected for a periplasmically exposed protein. Collapse of the pmf either by osmotic lysis of spheroplasts or addition of protonophores to spheroplasts, renders the amino terminal ⅔ of TonB resistant to exogenous proteinase K. It was not known, however, if this conformation was a “dead-end” representing a now permanently inactivated TonB or a temporary stall with the potential of resuming its energy transduction cycle. To distinguish between these possibilities, the reversibility of the TonB proteinase K resistant conformation was examined by washing away the previously added protonophore prior to proteinase K treatment. A “dead end” conformation would still be present after re-establishing pmf, while a stalled conformation would continue the cycle of TonB conformational changes and once again become susceptible to proteinase K. As observed previously, TonB in whole cells was not accessible by proteinase K but was fully accessible and proteolytically degraded in spheroplasts (W3110 sph), (Larsen et al., 1999, Mol. Microbiol. 31, 1809-1824). In spheroplasts where pmf was collapsed by addition of CCCP, treatment with proteinase K resulted in detection of the previously observed proteinase K resistant conformation of TonB. When CCCP was washed away and pmf restored, TonB again became completely sensitive to proteinase K. To determine if a functional conformation of TonB had indeed been restored, washed spheroplasts were re-treated with CCCP and then with proteinase K. Notably, the proteinase K resistant form of TonB was again detected. This indicated that TonB conformation was reversibly stalled in the absence of pmf and could recover when pmf was restored. ExbD also showed a reversible pmf-dependent change in proteinase K sensitivity and results are discussed below.

In vivo pmf-dependent TonB-ExbD interaction also occurs in spheroplasts. Spheroplast generation leaves the CM intact but, through disruption of the OM and hydrolysis of the peptidoglycan layer, exposes the periplasmic domains of CM proteins such as TonB and ExbD to solution. While the proteinase K assay takes advantage of this fact, this also equates to a non-native environment for these proteins, raising the question of whether this change in environment alters the native behavior or conformations of the solution-exposed domains. In vivo, TonB and ExbD periplasmic domains undergo an energized heterodimeric interaction, characterized by detection of a formaldehyde crosslinked heterodimer only in the presence of pmf (Ollis et al., 2009, Mol Microbiol 73, 466-81).

To address whether the periplasmic domains of TonB and ExbD in spheroplasts exhibit native conformations, spheroplasts were generated from a wild-type (W3110) strain and crosslinked with formaldehyde in the presence or absence of pmf (presence of CCCP). As observed previously for ExbD in whole cells, ExbD in spheroplasts crosslinked into homodimers and heterodimeric complexes with TonB or ExbB Importantly, the TonB-ExbD complex was pmf-dependent—it was not detected in CCCP-treated spheroplasts—demonstrating native TonB and ExbD periplasmic domain conformations in spheroplasts. The same held true for TonB, where all known complexes detected in formaldehyde-treated whole cells were detected in spheroplasts. This even included the TonB complexes with the OM proteins FepA and Lpp, likely due to fragments of the OM still attached to spheroplasts.

Spheroplast formation was confirmed by prominent detection of the TonB proteinase K resistant form in a portion of the CCCP-treated spheroplasts that were treated with proteinase K for 15 minutes prior to crosslinking. Two higher bands were also observed in this sample. One migrated at the apparent size of full-length TonB and was likely residual, undigested TonB. The highest band migrated at approximately 47 kDa. Based on its similar abundance to the TonB-ExbB crosslink, this was potentially a partially digested form of that complex. Its identity was not confirmed.

ExbD D25N mimics the effect of pmf collapse on TonB conformation. Previous work established that the presence of ExbD is required for the proteinase K resistant conformation of TonB. Only two point mutations are known to inactivate ExbD, D25N in the TMD and L132Q in the periplasmic domain. ExbD D25N lacks the highly conserved aspartate residue involved in response of ExbD paralogues MotB and TolR to pmf. The role of L132 in ExbD activity is unknown. Both ExbD D25N and L132Q, like wild-type ExbD, are stable proteins, capable of forming homodimers and heterodimers with ExbB, suggesting these point mutations do not significantly alter native ExbD conformation. They differ from wild-type ExbD only in their inability to form the pmf-dependent TonB-ExbD heterodimer (Ollis 2009, Mol Microbiol 73, 466-81). The effect of an ExbD mutant, where ExbD was present but not active, had not previously been examined for the proteinase K resistant conformation of TonB.

In these studies, ExbD D25N supported only a low level of the TonB proteinase K resistant conformation when immunoblotted after 15 min of proteinase K treatment in the presence of CCCP. Surprisingly, this conformation was present even in the absence of CCCP. This result suggested that, in the presence of ExbD D25N, the proteinase K resistant conformation of TonB could form regardless of pmf. This result was the first time the proteinase K resistant conformation of TonB was observed when pmf was present. This conformation of TonB was not detected in lysed spheroplasts expressing ExbD D25N, likely due to release of cytoplasmic proteases as seen previously. To better understand the behavior of TonB under these circumstances, the length of the proteinase K treatment was limited. Interestingly, wild-type ExbD supported a low level of the TonB proteinase K resistant conformation in the presence of pmf at early time points. As in the standard assay, by 15 min proteinase K treatment time, TonB was degraded. In the presence of CCCP, strong conversion of TonB to the proteinase K resistant conformation occurred by 2 min and was stable at least for 15 min ExbD D25N supported equally strong formation of the TonB proteinase K resistant form at early time points in both the absence and presence of CCCP. In contrast to the presence of wild-type ExbD, this fragment of TonB was rapidly proteolyzed by proteinase K under both conditions, as was ExbD D25N itself. The conformation could form, but the resistance to proteinase K was not maintained. In the early time points of proteinase K treatment a slightly larger band was also apparent and diminished as the treatment proceeded. It was also seen in the presence of ExbD L132Q and TonB H20A. The band was also faintly detected on longer exposures of the wild-type samples. This was likely another proteolytic product on the pathway to conversion of TonB to the final proteinase K resistant form.

The ExbD periplasmic domain was a likely candidate for involvement in the formation of the TonB proteinase K resistant form, which encompasses the amino terminal ⅔ of TonB and thus includes about 60% of the TonB periplasmic domain. In contrast to TMD mutant ExbD D25N, ExbD L132Q did not support the proteinase K resistant conformation of TonB, even if treatment time was reduced to 2 minutes. At 15 minutes, TonB in the presence of ExbD L132Q was fully sensitive to proteinase K, identical to the absence of ExbD (ΔexbD, ΔtolQR). Both ExbD D25N and ExbD L132Q assemble normally with ExbB. Thus it appeared that, as long as the periplasmic domain of ExbD was intact, it was possible to form the TonB proteinase K resistant conformation.

ExbD conformation changes when pmf is collapsed. ExbD is hypothesized to somehow convert the TonB carboxy terminus to an active conformation that initiates substrate transport through the OM transporters (Ollis et al., 2009, Mol Microbiol 73, 466-81). Since TonB does not have the capacity to directly respond to pmf, the possibility that ExbD was the source of the conformational response to pmf was investigated. ExbD is sensitive to proteolysis by proteinase K or trypsin in spheroplasts. When samples from the limited proteolysis studies of TonB conformational changes were immunoblotted with ExbD-specific antibodies, ExbD reversibly exhibited sensitivity or resistance to proteinase K, dependent upon the presence of pmf. Unlike TonB, the full length of ExbD became resistant to proteinase K when pmf was collapsed. While some variation was seen in the total level of ExbD that was proteinase K resistant, ExbD was consistently more resistant to proteinase K after collapse of pmf than when pmf was present. After 15 min of proteinase K treatment, ExbD D25N and L132Q substitutions exhibited almost full sensitivity in intact spheroplasts, similar to wild-type ExbD. In contrast to wild type ExbD, these mutants remained sensitive in the absence of pmf. Examination of proteinase K sensitivity at earlier time points showed each ExbD mutant was resistant to proteinase K at 2 min. Both ExbD mutants, however, were almost fully degraded by 15 minutes. These substitutions, in two different domains of ExbD, appeared to render it conformationally unresponsive to changes in pmf. The reasons for sensitivity could be different for each.

A wild-type TonB TMD is required for pmf-dependent ExbD conformational change. TonB H20A inactivates TonB. As seen previously for other TonB TMD mutants, TonB H20A itself was fully degraded by 15 min of proteinase K treatment in the presence of CCCP. When assayed at 2 min, a very faint band of a TonB H20A proteinase K resistant form was detected. Either a small fraction could achieve this conformation or the conformation was very unstable. The somewhat unexpected effect of TonB H20A or a tonB deletion on ExbD was to render it sensitive to proteinase K at both 15 min and 2 min. These data suggested that the conformational changes observed for both ExbD and TonB were somehow mutually interdependent.

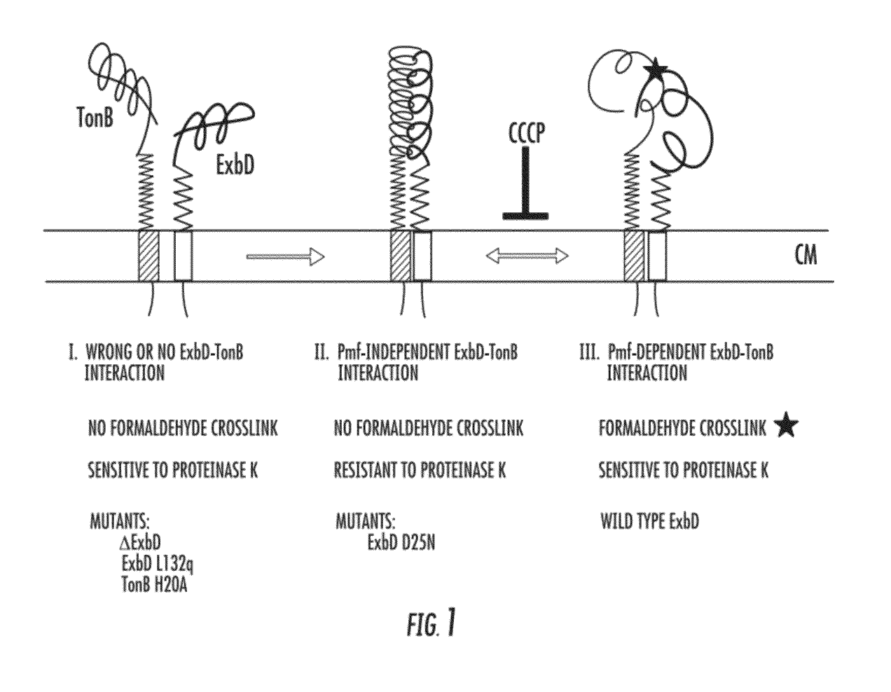

FIG. 1 shows a model for initial stages in TonB energization. Stage I is a theoretical possibility not demonstrated to exist for wild-type strains but invoked due to the behavior of ExbD periplasmic mutants and TonB in the absence of ExbD or vice versa. In Stage I, ExbD and TonB periplasmic domains are not in detectable contact. They cannot proceed to Stage II if ExbD carries periplasmic domain deletions in the region from 62-141 or the L132Q mutation. In this condition, TonB is fully sensitive to proteinase K. TonB carrying the H20A mutation also appears to be stalled at this Stage. In Stage II, the periplasmic domains of TonB and ExbD interact in a configuration that does not require the pmf. This configuration becomes detectable when TonB fails to proceed further to Stage III and remains stalled at Stage II, the hallmark of which is proteinase K resistance of the amino terminal ⅔ of TonB and of ExbD. Collapse of the pmf by CCCP or mutations in the ExbD amino terminus prevent ExbD and TonB from proceeding to Stage III. In Stage III, the conformational relationship between the TonB and ExbD periplasmic domains has changed such that formaldehyde crosslinkable residues on both proteins move into close proximity (star). This new conformational relationship is also marked by complete TonB sensitivity to proteinase K. The transition between Stages II and III is reversible, with presence or absence of pmf acting as the toggle switch.

In this model, it is proposed that TonB has three conformational states on its pathway to becoming “energized” by the pmf. Two of these states are sensitive to proteinase K and were thus a source of confusion in the original interpretation of this assay. The first stage is a currently hypothetical stage of unassembled ExbD and TonB periplasmic domains, represented by mutants with either an apparent lack of periplasmic domain TonB-ExbD interaction (ExbD L132Q) or inability to assemble (ΔexbB, ΔexbD, and possibly TonB H20A) (FIG. 1, Stage I). TonB proteinase K sensitivity in both the presence and absence of CCCP is diagnostic of inactive TonB stalled at this stage. Second is an assembled, pmf-independent interaction of TonB and ExbD periplasmic domains (FIG. 1, Stage II). This stage is characterized by a proteinase K resistant conformation that is only revealed when TonB is stalled at this stage by collapse of pmf or when TonB conformation cannot be coupled to the pmf, i.e. when ExbD D25N is present. This initial contact between TonB and ExbD periplasmic domains is almost certainly mediated through ExbB since it appears to be the scaffold that stabilizes both proteins, and the conformation is not detected if ExbB is deleted, i.e. TonB is stalled at Stage I. Third is energized TonB (FIG. 1, Stage III).

### Materials and Methods

Bacterial strains and plasmids. Bacterial strains and plasmids used in this study are listed in Table 1. pKP1333, pExbD(L132Q), was a derivative of pKP999 (exbD in pPro24). The L132Q substitution was generated using 30-cycle extra-long PCR. Forward and reverse primers were designed with the desired base change flanked on both sides by 12-15 homologous bases. DpnI digestion was used to remove the template plasmid. To ensure no unintended base changes were present, the sequence of the entire exbD gene was verified by DNA sequencing.

Media and culture conditions. Luria-Bertani (LB) and M9 minimal salts were prepared. Liquid cultures and agar plates were supplemented with 34 μg m1⁻¹ chloramphenicol or 100 μg m1⁻¹ ampicillin and plasmid-specific levels of sodium propionate or L-arabinose (percent as w/v), as needed for expression of ExbD and TonB proteins from plasmids. M9 salts were supplemented with 0.5% glycerol, 0.4 μg m1⁻¹ thiamine, 1 mM MgSO₄, 0.5 mM CaCl₂, 0.2% casamino acids, 40 μg m1⁻¹ tryptophan, and 1.85 μM or 37 μM FeCl₃·6H₂O. Cultures were grown with aeration at 37° C.

Proteinase K accessibility assays. For reversibility assays, spheroplasts were generated from wild-type cells (W3110) and treated with CCCP to collapse pmf. Identical results were observed when spheroplasts were treated with the protonophore DNP. Samples were harvested and treated with proteinase K or left untreated. Those samples represented the end point of the previous proteinase K assays, where treatment with proteinase K resulted in detection of the novel proteinase K resistant form of TonB. To re-establish pmf for the remaining sample, the proteinase K untreated, CCCP-treated spheroplasts were subsequently pelleted, washed, and resuspended in buffer without CCCP. These washed spheroplasts were divided in half. One sample was re-treated with CCCP, and one was treated with solvent only (DMSO). Both were then treated with proteinase K. As a control, whole cells were also treated with proteinase K or left untreated. All manipulations occurred at 4° C.

For standard assays, spheroplasts were prepared and treated with proteinase K as described previously (Larsen et al, 1999, Mol. Microbiol. 31, 1809-1824). The effect of protonophores was examined after treatment of spheroplasts with 60 μM carbonylcyanide-m-chlorophenylhydrazone (CCCP) or 10 mM DNP compared to an equal volume of solvent only, dimethyl sulfoxide (DMSO). After limited proteolysis, TCA precipitated samples were visualized on immunblots of 11% or 15% SDS polyacrylamide gels with TonB-specific monoclonal antibodies or ExbD-specific polyclonal antibodies, respectively. All manipulations occurred at 4° C.

Formaldehyde crosslinking in spheroplasts. Spheroplasts were prepared as above for standard assays and treated with DMSO solvent or 60 μM CCCP. A CCCP-treated sample was also treated with proteinase K for 15 min as described above. For formaldehyde crosslinking, spheroplasts were pelleted and resuspended in 100 mM sodium phosphate buffer, pH 6.8 containing 0.25M sucrose and 2 mM MgSO₄. For CCCP-treated spheroplasts, CCCP was maintained in the crosslinking buffer. Samples were treated with 1% monomeric paraformaldehyde (inverting to mix) for 15 min at room temperature. Pellets were solubilized at 60° C. in Laemmli sample buffer.

TABLE 1
Strains and plasmids used in this study.

| Strain or | | |
|---|---|---|
| Plasmid | Genotype or Phenotype | Reference |
| Strains | | |
| W3110 | F⁻ IN(rrnD-rrnE)1 | Hill, C. W. & Harnish, B. W. |
| | | 1981, Proc. Natl. |
| | | Acad. Sci. USA 78, |
| | | 7069-7072 |
| RA1045 | W3110, ΔexbD, ΔtolQR | Brinkman, K. K. & |
| | | Larsen, R. A. 2008, J |
| | | Bacteriol 190, 421-7 |
| KP1344 | W3110 tonB, P14::blaM | Larsen et al., 1999, Mol. |
| | | Microbiol. 31, 1809-1824 |
| Plasmids | | |
| pPro24 | Sodium propionate (2-methyl | Lee, S. K. & Keasling, J. D. |
| | citrate)-inducible, pBR322 ori | 2005, Appl Environ |
| | | Microbiol 71, 6856-62 |
| pKP381 | TonB H20A | Larsen et al., 2007, J |
| | | Bacteriology 189, 2825-2833 |
| pKP999 | exbD in pPro24 | Ollis et al., 2009, Mol |
| | | Microbiol 73, 466-81 |
| pKP1064 | ExbD D25N | Ollis et al., 2009, Mol |
| | | Microbiol 73, 466-81 |
| pKP1333 | ExbD L132Q | Present study |

### Example 2

### The ExbD Periplasmic Domain Contains Distinct Functional Regions for Two Stages in TonB Energization

The TonB system of Gram negative bacteria energizes active transport of diverse nutrients through high-affinity TonB-gated outer membrane transporters using energy derived from the cytoplasmic membrane protonmotive force. Cytoplasmic membrane proteins ExbB and ExbD harness the proton gradient to energize TonB, which directly contacts and transmits this energy to ligand-loaded transporters. In Escherichia coli, the periplasmic domain of ExbD appears to transition from protonmotive force-independent to protonmotive force-dependent interactions with TonB, catalyzing the conformational changes of TonB. A ten-residue deletion scanning analysis showed that while all regions except the extreme amino terminus of ExbD were indispensable for function, distinct roles for the amino and carboxy terminal regions of the ExbD periplasmic domain were evident. Like ExbD transmembrane domain residue D25, periplasmic residues 42-61 were essential for the conformational response of both ExbD and TonB to protonmotive force. Periplasmic residues 62-141 were required for proper assembly with the periplasmic domain of TonB, which does not require protonmotive force. Residues 92-121 were important for all three interactions previously observed for formaldehyde-crosslinked ExbD: ExbD homodimers, TonB-ExbD heterodimers, and ExbD-ExbB heterodimers. This latter result raised the question of whether interactions might occur between the ExbD periplasmic domain and the few residues of ExbB known to occupy the periplasm.

Here we provide the first detailed insights into functional domains of the Escherichia coli ExbD protein using a 10-residue deletion scanning analysis. This “global” mutagenesis approach identified two regions of the ExbD periplasmic domain with distinct functional roles that might have been missed by directed mutagenesis studies of individual residues. The region from ExbD residues 42-61 was required for TonB to progress to its energized Stage III conformation, but not initial interaction with TonB that occurs in Stage II. No known ExbD missense mutations have previously directed focus to this region of ExbD. In addition, the region from residues 62-141 (the carboxy terminus) was important for proper assembly with TonB (Stage II), and included a 30 residue subdomain that was important for all known ExbD protein-protein interactions.

### Materials and Methods

Bacterial Strains and Plasmids

Strains and plasmids used in this study are listed in Table 2. KP1522 was constructed by P1vir transduction of ΔexbD::cam from RA1021 into RA1016, creating W3110, ΔexbD::cam, ΔtolQRA.

A set of ExbD 10 amino acid deletion variants was constructed where the exbB, exbD operon was encoded on the plasmid. Plasmids pKP724 and pKP761 through pKP764 were constructed by in-frame deletion of ten exbD codons using extra-long PCR, as previously described (Higgs et al., J Bacteriol 180:6031-8). All were derivatives of pKP660 (Ollis et al., Mol Microbiol 73:466-81). Sequences of exbB and exbD were confirmed by DNA sequencing. A second set of ExbD deletion variants, pKP1246 through pKP1259, was constructed where the exbB gene was not present on the plasmid. pKP1246 through pKP1259 were derivatives of the first set of plasmids that included exbB. The second set was constructed by extra-long PCR to create an in-frame deletion of exbB. Due to a possible requirement for translational coupling between exbB and exbD, deletion of exbB left intact the initiating ATG plus last 25 codons of exbB. Sequences of the exbB segment and exbD gene were confirmed by DNA sequencing.

pKP920, which expresses only ExbB, was also a derivative of pKP660. Extra-long PCR was used to delete exbD from its ATG start codon through 6 bases following the exbD TAA stop codon. The sequence of exbB was confirmed by DNA sequencing.

pKP1194, exbD in pBAD24, was constructed by digestion of pKP999 (exbD in pPro24) and pBAD24 with NcoI. Fragments were separated by gel electrophoresis. The 4542 bp fragment of pBAD24 and 506 bp fragment of pKP999, containing exbD, were purified by gel extraction and ligated together after treatment of the vector fragment with Antarctic Phosphatase. Proper orientation of the insert was verified by FspI digestion. The exbD sequence in pBAD24 was confirmed by DNA sequencing.

Induction levels for ExbD deletion variants: For assays in T broth (spot titers), the following percentages of arabinose were added at subculture to induce expression of ExbD variants near native levels of ExbD: pKP660=no inducer, pKP761=0.0001%, pKP760=0.0003%, pKP759=0.05% glucose (to repress basal levels of overexpression), pKP758=0.0001%, pKP762=0.0003%, pKP757=0.001%, pKP756=0.0025%, pKP755=0.001%, pKP754=0.004%, pKP753=0.006%, pKP752=0.008%, pKP763=0.006%, pKP764=0.004%, pKP724=0.002%.

For assays in 1×M9, 37 μM Fe (proteinase K accessibility, [⁵⁵Fe]-ferrichrome uptake, formaldehyde crosslinking, and sucrose density gradient fractionation), the following percentages of arabinose were added at subculture to induce expression of ExbD variants near native levels of ExbD: pKP660=no inducer, pKP761=0.0008%, pKP760=0.0008%, pKP759=0.3% glucose (to repress basal levels of overexpression), pKP758=0.002% glucose (to repress basal levels of overexpression), pKP762=0.0006%, pKP757=0.0007%, pKP756=0.006%, pKP755=0.008%, pKP754=0.15%, pKP753=0.18%, pKP752=0.2%, pKP763=0.18%, pKP764=0.2%, pKP724=0.006%.

Activity assays: Spot titer assays were performed. Initial rates of [⁵⁵Fe]-ferrichrome uptake were determined.

In vivo formaldehyde crosslinking: Saturated overnight cultures were subcultured 1:100 into M9 minimal media (above) supplemented with L-arabinose. At mid-exponential phase, cells were treated with formaldehyde. Crosslinked complexes were detected by immunoblotting with ExbD-specific polyclonal antibodies or TonB-specific monoclonal antibodies. To normalize levels of ExbD monomer after crosslinking, the following ODmL were loaded on the SDS-polyacrylamide gel: W3110=0.2, RA1017/pKP660=0.25, RA1017/pKP761=0.2, RA1017/pKP760=0.4, RA1017/pKP759=0.15, RA1017/pKP758=0.2, RA1017/pKP762=0.2, RA1017/pKP757=0.2, RA1017/pKP756=0.5, RA1017/pKP755=0.35, RA1017/pKP754=0.4, RA1017/pKP753=0.5, RA1017/pKP752=0.45, RA1017/pKP763=0.45, RA1017/pKP764=0.4, RA1017/pKP724=0.42.

### Results

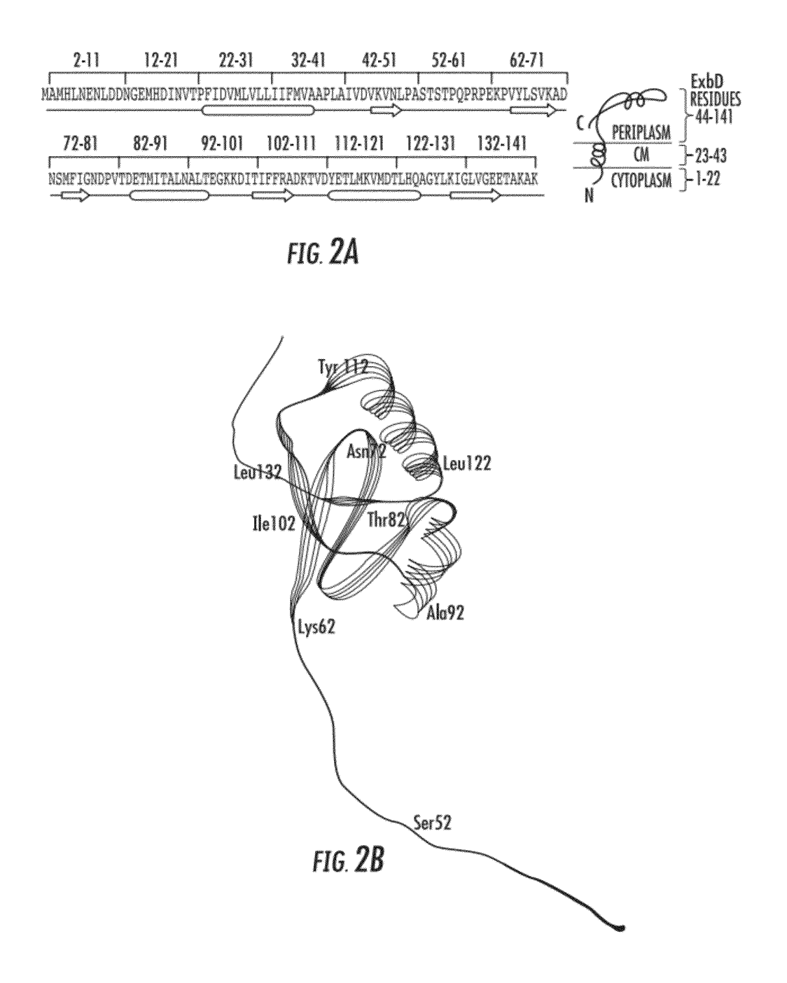

The ExbD periplasmic domain is important for ExbD stability and activity: With the exception of ExbD D25N and L132Q, which are inactive, no comprehensive studies have defined functionally important regions of ExbD. A 10 residue deletion analysis approach previously identified several dispensible regions of the ExbD paralogue MotB immediately following the TMD, suggesting this method could be used to consider an entire protein and focus in on important regions. To define functional regions of ExbD, 14 consecutive 10 amino acid deletions were constructed along the length of the ExbD from residues 2-21 (cytoplasmic amino terminus), 22-41 (TMD is predicted to be 23-43), and 42-141 (periplasmic carboxy terminus) (FIG. 2A). Six of the periplasmic domain deletions, from 62-131, corresponded with a region of defined tertiary structure on the ExbD NMR structure but were not based on it (Garcia-Herrero et al., Mol Microbiol 66:872-89) (FIG. 2B). The NMR structure was determined in the absence of both the ExbD TMD and protonmotive force, both of which are essential for ExbD function and conformational response to protonmotive force. While it is unknown if the NMR structure represents a native conformation of ExbD, in vivo studies of TonB-ExbD interactions indicate that the ExbD carboxy terminus is conformationally dynamic.

In this study, plasmid encoded exbD deletion variants were expressed under control of the arabinose promoter. ExbB, which is the first gene in the operon with exbD, was present on the chromosome of KP1522 under control of its native promoter, and the chromosomal exbD gene was deleted. For activity assays, attempts were made to express all ExbD deletions at levels equal to native, chromosomally-encoded ExbD. For deletions in the amino terminus or TMD, chromosomal levels could be achieved; deletions in the TMD region appeared to be highly stable. However, 8 of the periplasmic deletions covering the region from 62-141 were so proteolytically unstable that expression levels equal to that of chromosomally-encoded ExbD could not be achieved with even the highest concentration of arabinose.

The same set of deletion variants was then re-constructed on plasmids encoding both exbB and exbD under control of the arabinose promoter. With the concomitant high level of ExbB expression, the subset of ExbD periplasmic domain ten amino acid deletions from residues 82-131 could be expressed at chromosomally encoded ExbD levels (Methods and Materials). In subsequent experiments the levels of ExbB varied considerably as different levels of inducer were used to achieve chromosomal levels of ExbD deletions that had varying levels of proteolytic susceptibility.

Spot titers, which are capable of detecting very low levels of TonB activity, measure sensitivity to colicins and bacteriophage that enter and subsequently kill E. coli via the TonB system. 13 of the 14 mutants showed complete tolerance (insensitivity) to TonB-dependent colicins and bacteriophage (Table 3). Only ExbDΔ2-11 was active, supporting essentially full sensitivity to colicins (Table 3) and transporting ⁵⁵Fe-ferrichrome at a rate near that of wild-type, plasmid-encoded ExbD (Table 4). Consistent with their insensitivity to phage and colicins, the other 13 deletion variants supported no iron transport.

Identification of an ExbD periplasmic domain required to energize TonB: ExbD is an essential protein for the stages leading to TonB energy-dependent conformational changes. TonB and ExbD form an initial protonmotive force-independent complex that renders both proteins resistant to exogenous proteinase K in spheroplasts. The apparent action of protonmotive force is subsequently to promote the rearrangement of the initial TonB-ExbD periplasmic interactions such that both proteins exhibit sensitivity to proteinase K. The proteinase K resistant forms of TonB and ExbD can be observed in two ways: either by treating cells with protonophores to stall the TonB-ExbD interaction at the protonmotive force independent stage or when ExbD D25N is present.

The ExbD deletions were surveyed for their ability to support the TonB proteinase K resistant conformation indicative of protonmotive force-independent ExbD-TonB periplasmic domain interaction. Like wild-type ExbD, ExbD Δ2-11 fully supported formation of the TonB proteinase K resistant conformation after collapse of the protonmotive force by addition of the protonophore CCCP. ExbDΔ2-11, itself, exhibited sensitivity to proteinase K by 15 min but stable resistance after collapse of protonmotive force, also like wild-type ExbD.

The majority of inactive ExbD deletion variants resulted in complete sensitivity of TonB to proteinase K, both in energized spheroplasts and CCCP-treated spheroplasts. Sensitivity to proteinase K when protonmotive force is collapsed is a characteristic of TonB and ExbD that have not yet assembled into the protonmotive force independent complex, due either to the absence of ExbB or ExbD or the presence of mutations in either protein that prevent proper assembly. As expected the same result was obtained in the parent strain, RA1017 that is deleted for exbB/D.

The results with ExbD Δ42-51 and ExbDΔ52-61 were unique. Similar to the effect of an ExbD D25N mutation, both supported the proteinse K resistant conformation of TonB at 2 min, in the presence and absence of protonmotive force. In both cases the band was more susceptible to proteolysis, but not fully degraded, by 15 min in the presence of protonmotive force and maintained more stable resistance to proteinase K after collapse of protonmotive force. ExbD Δ42-51 and Δ52-61 themselves were resistant to proteinase K in spheroplasts under all conditions tested, similar to wild-type ExbD in the presence of CCCP.

Cytoplasmic deletion ExbD Δ12-21 supported a trace amount of the TonB proteinase K resistant conformation at only the earliest time point of 2 min Detection, though at a much lower level, was protonmotive force-independent like ExbDΔ42-51 and Δ52-61. This initial assembly of TonB with the periplasmic domain of ExbDΔ12-21 was perhaps unstable or inefficiently formed. Overall, the regions of 12-21, 42-61, and 62-141 were apparently important in different ways for ExbD interactions with TonB.

Inactive ExbD ten amino acid deletions exhibit changed protein-protein interaction: Since deletions within the domains of ExbD differentially affected initial assembly with TonB, the affects of deletions on other detectable interactions of ExbD were examined. ExbD formaldehyde crosslinks in vivo into homodimers, an ExbD-ExbB heterodimer, and an ExbD-TonB heterodimer. To determine which interactions were supported by the 10-residue deletions, their individual crosslinking profiles were analyzed. Cells expressing the deletion variants were treated with monomeric paraformaldehyde, resolved on SDS-polyacrylamide gels, and immunoblotted using polyclonal ExbD-specific antibody. As previously observed, the only active deletion, ExbDΔ2-11 formed all three known complexes at levels similar to wild-type ExbD.

None of the 13 inactive deletion variants formed a detectable crosslink to TonB, an interaction that, to date, appears to occur only through an active conformation of ExbD. In cases where a potential TonB-ExbD heterodimer was present based on a complex of similar molecular mass (Δ72-81, for example), it was ruled out because the crosslinking profile in the absence of TonB remained the same. ExbDΔ42-51 and Δ52-61 profiles, which clearly lacked the TonB-ExbD heterodimer, were also identical with and without TonB. This confirmed that while the inactive ExbD deletions could interact with TonB to support formation of the protonmotive force-independent proteinase K resistant conformation, as shown above, they did not form the protonmotive force-dependent ExbD-TonB formaldehyde crosslink. ExbDΔ12-21 exhibited decreased levels of crosslinking to ExbB and homodimer formation. As expected, ExbDΔ22-31 and Δ32-41, each missing half of the proposed ExbD TMD formed no detectable complexes. The likelihood that absence of complexes for those deletions was due to their sequestration in the cytoplasm was investigated below.

The five deletions within the region from residues 42-91 could all be formaldehyde crosslinked to some degree with ExbB and form ExbD homodimers. Increased formation of the ExbB-ExbD complex was likely due in part to increased levels of ExbB in the strains where high levels of arabinose were required for expression of the ExbD variants near native ExbD levels. However, the relative level of complex formed did not correlate with the level of inducer in all cases. ExbDΔ42-51 and Δ52-61 mediated increased association with ExbB, especially compared with the active ExbDΔ2-11, even though at least 10× less inducer was used compared to the ExbD deletions in the region from 62-141 (see Materials and Methods for induction levels). ExbD Δ62-71 exhibited low levels of complex formation with ExbB that were about equal to its level of homodimer. ExbDΔ72-81 and Δ82-91 formed homodimers with high efficiency, but also required high levels of inducer that subsequently gave rise to high levels of ExbB.

Three deletion variants encompassing the region from residues 92-121 were unable to significantly form any of the expected complexes, including the crosslink to ExbB, a protein with only minor soluble periplasmic domains. Nonetheless, ExbB overexpression stabilized these deletion variants to the point where they could be detected by immunoblot, suggesting that some interactions with ExbB remained. Homodimer formation was weak for Δ92-101, and no detectable homodimers were trapped for Δ102-111 or Δ112-121. It was unlikely that this region affected ExbD export since the three deletions formed a formaldehyde crosslinked complex with an unknown protein. This unidentified complex was observed for most of the other deletions, each of which had been properly exported based on their ability to form complexes with ExbB and was not detected with either export-deficient (see below) ExbD TMD deletion variant.

The remaining two deletion variants, Δ122-131 and Δ132-141, each complexed with ExbB and into homodimers. Δ122-131 showed increased homodimer formation and a strong complex with the unknown protein. Intensities of the ExbDΔ132-141 homodimer and complex with ExbB were comparable to those formed with wild-type ExbD.

Deletions within the ExbD TMD prevent CM insertion: The ExbD TMD is predicted to span residues 23-43, so two ExbD deletion variants, Δ22-31 and Δ32-41, each had partial potential TMDs (FIG. 2A). No formaldehyde crosslinked complexes were observed for either variant, suggesting these mutants were not properly inserted in the CM. To directly test their localization, strains expressing the TMD deletion variants ExbDΔ22-31 and Δ32-41 were fractionated on sucrose density gradients. Both variants fractionated with soluble proteins and were sensitive to exogenous proteinase K only after lysis of spheroplasts, indicating they were localized in the cytoplasm. A faint band of the stable degradation product of ExbD Δ22-31 was still resistant to 15 min proteinase K treatment in lysed spheroplasts. This may be a more proteolytically stable form of this deletion variant.

### Discussion

Although ExbD is an essential protein in the TonB system, it has not been subjected to a comprehensive mutagenesis study. We initiated our study of ExbD with 10-residue scanning mutagenesis, an approach predicted to cause major structural changes but which has previously demonstrated utility in identifying regions dispensable, or not, for protein function (Muramoto, K., and R. M. Macnab. Mol Microbiol 29:1191-202, 1998). While all 10 periplasmic domain 10-residue ExbD deletions were inactive, two different functional domains within the ExbD periplasmic domain could be identified. Residues from 62-141 were important for the protonmotive force-independent contacts between TonB and ExbD periplasmic domains, and the region from 42-61 was important for the subsequent conformational response of assembled TonB-ExbD heterodimers to protonmotive force.

A functional unit consisting of the ExbD amino terminal periplasmic domain and TMD: While ExbD Δ42-51 and Δ52-61 were fully capable of initial assembly with TonB, they were blocked in the transition to an energized TonB-ExbD interaction. These mutants exhibited similar phenotypes to those previously observed with ExbD D25N in all assays, where TonB conformation remains stalled at Stage II. Thus it appeared that the TMD and the region immediately following it were directly involved in response of ExbD, and consequently TonB, to protonmotive force. ExbD residues 44-66 are disordered in the NMR solution structure of the periplasmic domain, and the same region from residues 45-66 was also predicted to be disordered by PONDR™ analysis. TonB residues 102-151 are disordered in the solution structure, with an even larger uninterrupted region of residues ˜35-170 predicted to be disordered by PONDR™. One possibility is that the disordered region of ExbD is important for the carboxy terminus of ExbD to achieve the conformation that allows it to energize TonB, with residues 45-66 serving to propagate changes from the TMD to the structured carboxy terminus of ExbD, which is involved in direct interaction with the TonB periplasmic domain. Alternatively, the disordered regions of TonB and ExbD may need to find each other and collapse into a defined structure for TonB to be correctly energized.

The Conformation of ExbD Residues 62-141 is Important for Assembly with TonB:

Protonmotive force-independent assembly of the ExbD and TonB periplasmic domains (Stage II) is an essential stage prior to the action of the protonmotive force. The entire ExbD carboxy terminus from residue 62-141 appeared to be important for that initial assembly with TonB, and all deletions within this region stalled TonB at Stage I. The 8 deletions from residues 62-141 were highly unstable, suggesting that 10-residue deletions within this region had greater structural ramifications than those from residues 42-61. Consistent with that idea, the carboxy terminal deletions encompassed the region of defined tertiary structure (residues 64-133) in the ExbD periplasmic domain NMR structure (FIG. 2B). However, the conformation of at least 5 deletions within this region, from residues 62-91 and 122-141, was not so distorted as to prevent formation of an ExbD homodimer, a known biologically relevant interaction, previously detected in complex with ExbB in vivo. None of the deletions within this span, however, were observed here to interact with TonB. Thus it appeared that ExbD homodimer formation had less stringent structural requirements than those for initial ExbD-TonB assembly.

Inactivity due to deletion of the last 10 amino acids of ExbD, Δ132-141, may be an effect of removal of L132, previously shown to be important for ExbD activity and assembly of ExbD and TonB periplasmic domains. Previous work has described almost full activity of a construct of ExbD fused at residue 134 to β-lactamase. This might suggest at least the last 7 residues of ExbD are dispensable, with the important function of this region coming from L132.

Determinants of ExbD-ExbB formaldehyde crosslinks: Three deletions within the ExbD carboxy terminus, from residues 92-121 lacked the ability to form all three known ExbD formaldehyde-crosslinked complexes: ExbD homodimers, ExbD-TonB heterodimers, and ExbD-ExbB heterodimers. An inability to form normally stabilizing interactions could contribute to the observation that these three deletions were also the most proteolytically unstable. We showed previously that residues within this span are involved in direct homodimer and TonB-ExbD heterodimer formation, and it was this study that led us to target that region. The requirement of residues 92-121 for formaldehyde crosslinked interaction with ExbB was unexpected, since the majority of ExbB residues are localized to the cytoplasm. There is currently no evidence for direct interaction of the ExbD periplasmic domain with ExbB, however periplasmic domain interaction at the CM has been proposed between homologous proteins in the Mot and Tol protein systems. The potential for periplasmic domain interaction between ExbD and ExbB and a role for such interaction has yet to be explored. ExbDΔ42-51 and Δ52-61 exhibited increased formaldehyde crosslinking with ExbB, suggesting that this region might be important for subsequent conformational changes that released ExbD from ExbB contact.

Commonalities and differences with MotB: MotB, a paralogue of ExbD, is tolerant of the deletion of 5 successive 10-residue spans immediately following its TMD. In contrast, 10-residue deletions at all sites in ExbD except the extreme amino terminus resulted in complete inactivity. This raises the question of how mechanistically similar ExbD and MotB are. The periplasmic domains of ExbD and MotB are entirely dissimilar in sequence, including the fact that ExbD lacks the peptidoglycan binding domain of MotB. The MotB periplasmic domain is also more than twice the size of this domain of ExbD. However, the general architecture of the proteins is similar in that each contains a highly conserved TMD with an essential aspartate, a periplasmic carboxy terminus that appears to define functional interactions of each protein, and a flexible region connecting these two domains. It may be that the periplasmic domains of MotB and ExbD have functionally diverged but common elements of the mechanism of harnessing energy derived from the protonmotive force remain.

Pivotal roles for flexible regions of the periplasmic domain, adjacent to the TMD, in propagating conformational changes between the TMD and carboxy terminus may be a conserved mechanism between MotB and ExbD. In MotB, bi-directional signaling is proposed based primarily on crystal structures of the H. pylori His₆-MotB periplasmic domain containing various truncations of a linker region (residues 64-112). It is currently unknown whether the ExbD region from 42-61 is important for TMD conformation, or vice versa, though it is clear the region is important for conformational response of ExbD to the protonmotive force.

Identification of cytoplasmic residues important for ExbD function: Reasons for the complete inactivity caused by deletion of the second half of the ExbD cytoplasmic domain, Δ12-21, were unclear. This span of residues may be important for proper or stable assembly of ExbD, since ExbDΔ12-21 exhibited weak protein-protein interactions, evidenced by both formaldehyde cros slinking and a low level of the TonB proteinase K resistant conformation. ExbD Δ12-21 also failed to support the conformational response of TonB to protonmotive force, as the level of the proteinase K resistant TonB was unchanged after collapse of protonmotive force. Recently, residues in the cytoplasmic carboxy terminus of ExbB, where contact with the cytoplasmic amino terminus of ExbD is possible, were also shown to be important for response of the ExbD and TonB periplasmic domains to protonmotive force. Residues 12-17 are not essential for ExbD function since ExbDΔ4-15, ExbD H16A, and ExbD D17A retain activity. Therefore, residues 18-21 make an important but currently unknown contribution to ExbD function.

In summary, this comprehensive deletion analysis identified two different domains of the ExbD periplasmic domain, with a clear functional separation between the residues immediately following the ExbD transmembrane domain and the carboxy terminal 80 residues. A subdomain, residues 92-121, previously known to be important for interaction with TonB or another ExbD, was also found to be important in supporting ExbD-ExbB interaction. The importance of the overall conformation of the ExbD periplasmic domain, or potential to achieve multiple conformations, is in accordance with a role in regulating TonB conformation. It will be important to further determine how these specific regions of the ExbD periplasmic domain function in the energization of TonB and the direction of apparent signal propagation between functional domains of ExbD.

TABLE 2
Strains and plasmids used in this study.

| Strain or Plasmid | Genotype or Phenotype |
|---|---|
| Strains | |
| W3110 | F⁻ IN(rrnD-rrnE)1 |
| RA1016 | W3110, ΔtolQRA |
| RA1017 | W3110 ΔexbBD::kan, ΔtolQRA |
| RA1021 | W3110, ΔexbD::cam |
| KP1503 | GM1, exbB::Tn10, tolQₐₘ, ΔtonB::kan |
| KP1522 | W3110 ΔexbD::cam, ΔtolQRA |
| Plasmids | |
| pBAD24 | L-arabinose-inducible, pBR322 ori |
| pPro24 | Sodium propionate (2-methyl citrate)- |
| | inducible, pBR322 ori |
| pKP920 | exbB in pBAD24 |
| pKP999 | exbD in pPro24 |
| pKP660 | exbB, exbD in pBAD24 |
| pKP761 | ExbB, ExbDΔ2-11 |
| pKP760 | ExbB, ExbDΔ12-21 |
| pKP759 | ExbB, ExbDΔ22-31 |
| pKP758 | ExbB, ExbDΔ32-41 |
| pKP762 | ExbB, ExbDΔ42-51 |
| pKP757 | ExbB, ExbDΔ52-61 |
| pKP756 | ExbB, ExbDΔ62-71 |
| pKP755 | ExbB, ExbDΔ72-81 |
| pKP754 | ExbB, ExbDΔ82-91 |
| pKP753 | ExbB, ExbDΔ92-101 |
| pKP752 | ExbB, ExbDΔ102-111 |
| pKP763 | ExbB, ExbDΔ112-121 |
| pKP764 | ExbB, ExbDΔ122-131 |
| pKP724 | ExbB, ExbDΔ132-141 |
| pKP1194 | exbD in pBAD24 |
| pKP1246 | ExbDΔ2-11 |
| pKP1247 | ExbDΔ12-21 |
| pKP1248 | ExbDΔ22-31 |
| pKP1249 | ExbDΔ32-41 |
| pKP1250 | ExbDΔ42-51 |
| pKP1251 | ExbDΔ52-61 |
| pKP1252 | ExbDΔ62-71 |
| pKP1253 | ExbDΔ72-81 |
| pKP1254 | ExbDΔ82-91 |
| pKP1255 | ExbDΔ92-101 |
| pKP1256 | ExbDΔ102-111 |
| pKP1267 | ExbDΔ112-121 |
| pKP1258 | ExbDΔ122-131 |
| pKP1259 | ExbDΔ132-141 |

TABLE 3
Spot titer assay results

| | | Susceptibility^{a} | | | |
|---|---|---|---|---|---|
| Strain | Phenotype | φ80 | Colicin B | Colicin M | Colicin Ia |
| W3110 | wild type | 9, 9, 9 | 8, 8, 8 | 6, 6, 6 | 7, 8, 8 |
| RA1017 | ΔExbBD, ΔTolQRA | T, T, T^{b} | T, T, T | T, T, T | T, T, T |
| RA1017/pKP660 | ExbB, ExbD | 7, 7, 7 | 5, 5, 5 | 4, 4, 4 | 6, 6, 6 |
| RA1017/pKP761 | ExbB, ExbDΔ2-11 | 7, 7, 7 | 4, 4, 4 | 4, 4, 4 | 6, 6, 6 |
| RA1017/pKP760 | ExbB, ExbDΔ12-21 | T, T, T | T, T, T | T, T, T | T, T, T |
| RA1017/pKP759 | ExbB, ExbDΔ22-31 | T, T, T | T, T, T | T, T, T | T, T, T |
| RA1017/pKP758 | ExbB, ExbDΔ32-41 | T, T, T | T, T, T | T, T, T | T, T, T |
| RA1017/pKP762 | ExbB, ExbDΔ42-51 | T, T, T | T, T, T | T, T, T | T, T, T |
| RA1017/pKP757 | ExbB, ExbDΔ52-61 | T, T, T | T, T, T | T, T, T | T, T, T |
| RA1017/pKP756 | ExbB, ExbDΔ62-71 | T, T, T | T, T, T | T, T, T | T, T, T |
| RA1017/pKP755 | ExbB, ExbDΔ72-81 | T, T, T | T, T, T | T, T, T | T, T, T |
| RA1017/pKP754 | ExbB, ExbDΔ82-91 | T, T, T | T, T, T | T, T, T | T, T, T |
| RA1017/pKP753 | ExbB, ExbDΔ92-101 | T, T, T | T, T, T | T, T, T | T, T, T |
| RA1017/pKP752 | ExbB, ExbDΔ102-111 | T, T, T | T, T, T | T, T, T | T, T, T |
| RA1017/pKP763 | ExbB, ExbDΔ112-121 | T, T, T | T, T, T | T, T, T | T, T, T |
| RA1017/pKP764 | ExbB, ExbDΔ122-131 | T, T, T | T, T, T | T, T, T | T, T, T |
| RA1017/pKP724 | ExbB, ExbDΔ132-141 | T, T, T | T, T, T | T, T, T | T, T, T |

^{a}Scored as the highest ten-fold dilution of bacteriophage φ80 or five-fold dilution of a standard colicin preparation that provided an evident zone of clearing on a cell lawn.
^{b}“T” indicates tolerance to undiluted colicin or phage (no clearing of the lawn). The values of three platings are presented for each strain/plasmid and colicin or phage pairing. Expression of ExbD variants to near levels of chromosomally-encoded ExbD was verified by immunoblots with ExbD-specific antibodies.

TABLE 4
Transport of ⁵⁵Fe-loaded ferrichrome

| | | Initial Rate of | % Wild-type |
|---|---|---|---|
| Strain | Phenotype | Transport^{a} | Activity^{b} |
| KP1522 | ΔExbBD, | −4.167 ± 2.309 | 0 |
| | ΔTolQRA | | |
| KP1522/pKP1194 | ExbD | 672.2 ± 15.97 | 100 |
| KP1522/pKP1246 | ExbDΔ2-11 | 654.7 ± 11.55 | 97 |

^{a}Strains/plasmids indicated were assayed for ability to transport ⁵⁵Fe-loaded ferrichrome as described in Materials and Methods. Plasmid-encoded ExbD variants were induced with the following percentages of arabinose: pKP1194 = .005%, pKP1246 = .08%
^{b}Percent wild-type activity was recorded as the initial rate of transport of the variant strain divided by the initial rate of transport of the wild-type strain (multiplied by 100). Rate of transport by the strain expressing ExbDΔ2-11 was also compared to the rate supported by plasmid-encoded wild-type ExbD (in parentheses). Expression levels for plasmid-encoded ExbD and ExbDΔ2-11 equal to chromosomally-encoded ExbD (W3110) levels were confirmed by Western blot with ExbD-specific polyclonal antibodies (data not shown).

### Example 3

### The Same Periplasmic ExbD Residues Mediate in Vivo Interactions Between ExbD Dimers and ExbD-TonB Heterodimers

This study identified sites of in vivo homodimeric interactions within ExbD periplasmic domain residues 92-121. Mapping of dimer-forming cys substitutions onto the previously solved ExbD periplasmic domain solution structure suggested that in vivo the periplasmic domain was conformationally dynamic. ExbD was active as a homodimer but not through all cys substitution sites, confirming the importance of a conformationally dynamic ExbD periplasmic domain. The degree of ExbD dimer formation increased in the absence of TonB. ExbD disulfide-linked homodimers could be crosslinked in vivo to ExbB, but ExbB was not required for their formation. Most importantly, the majority of ExbD cys substitutions that mediated homodimer formation also mediated ExbD-TonB heterodimer formation with TonB A150C. These results suggest a model where ExbD forms homodimers, and following subsequent association with ExbB, exchanges partners to form an ExbD-TonB heterodimer.

In this study, cysteine scanning of a 30-residue region of the ExbD periplasmic domain identified regions involved in homodimer formation in vivo, some of which mapped to the region identified in the NMR studies and some of which mapped to the opposite end of the solution structure, suggesting that the ExbD TMD contributes substantially to the conformation of a dynamic ExbD carboxy terminus. Most importantly, the same set of ExbD cys substitutions that mediated spontaneous disulfide-linked homodimer formation also mediated spontaneous heterodimer formation with TonB A150C in vivo.

### Results

Cysteine substitutions between ExbD residues 92-121 form spontaneous disulfide-linked dimers.

As described in Example 2, a thirty-residue region, residues 92-121, was identified in the periplasmic domain of ExbD that appeared to be important in mediating protein-protein interactions of ExbD. The solution structure of the isolated ExbD periplasmic domain showed ExbD residues 104-116 were involved in homomultimeric interactions in vitro involving four to seven ExbD periplasmic domains (Garcia-Herrero et al., 2007, Molecular microbiology 66: 872-889). In vivo, ExbD A92C can be trapped in disulfide-linked homodimers (Ollis et al., 2009, Molecular microbiology 73: 466-481). To further investigate the role of this region of ExbD in homodimer formation in vivo, cysteine substitutions were individually constructed at each of the remaining 29 residues. ExbD has no native cysteine residues so wild-type ExbD encoded on plasmid pKP999 was used as the template.

Each plasmid-encoded substitution was expressed in strain RA1045 (ΔexbD, ΔtolQR), where deletion of tolQR prevented the activity attributable to crosstalk of TonB with this homologous system. Attempts were made to express each ExbD cys substitution at levels equal to native ExbD when analyzed under reducing conditions. Immunoblots with ExbD-specific polyclonal antibody of steady-state levels of TCA precipitated proteins resolved on SDS-polyacrylamide gels showed all but one of the substitutions could be stably expressed. ExbD I102C was proteolytically unstable and only faintly detected on longer exposures, even when induced with the highest concentration of sodium propionate. The instability was not specific to substitution with cysteine, since ExbD I102A was also proteolytically unstable. Cys substitutions at T94, K98, and D107 also decreased ExbD stability, but these substitutions could be expressed to native ExbD levels with higher concentrations of sodium propionate. ExbD L93C showed aberrant migration, migrating slower than wild-type ExbD. ExbD L93A also exhibited slower migration. Single amino acid substitutions can alter protein mobility on SDS-polyacrylamide gels.

To see if any of the ExbD periplasmic domain cys substitutions were capable of forming spontaneous disulfide-linked homodimers, samples were prepared under non-reducing conditions. Bacterial cell pellets were solubilized in sample buffer containing iodoacetamide to alkylate free cysteines and prevent dimer formation after cell lysis. ExbD homodimers would theoretically migrate at about 31 kDa. Accordingly, ExbD-specific immunoblots of non-reducing SDS-polyacrylamide gels showed a number of ExbD cys substitutions formed disulfide-linked homodimers, with the highest level of complex formation observed for G96C, D99C, 1102C, D107C, K108C, Y112C, and E113C. Notably, dimer formation appeared to stabilize ExbD I102C, as a dimer was detected even when no monomeric form was detected under reducing conditions. Lower levels of dimer formation were observed for A92C, E95C, K98C, T109C, V110C, D111C, M116C, and D120C. For a few substitutions, a higher complex was formed, migrating around 40 kDa and most significantly detected with G96C, K98C, and D99C. The identity of this complex is unknown.

For certain cys substitutions, the nature of the native side chain would not favor interaction with the same residue of another ExbD. Asp99 and Lys108, for example, both formed prominent homodimeric interactions when substituted with cys, but two side chains with the same charge are unlikely to interact. For these cys substitutions, trapped interaction serves as evidence that the regions of ExbD where these residues are located come into close contact in vivo, and sites of disulfide bonds are therefore referred to as regions of interaction.

ExbD residues 92 through 121 are tolerant to substitution with cysteine. The ability of each cys-substituted ExbD to support ferrichrome-mediated iron transport under non-reducing conditions was determined, with each substitution expressed to native ExbD levels. The corresponding degree of dimer formation for each strain assayed was also determined All 30 substitutions were active, with Y112C supporting the lowest initial rate of transport at about 80% the rate of wild-type plasmid-encoded ExbD.

The 80% activity of ExbD I102C, for which monomer was not detected, suggested that ExbD was active as a homodimer, and at levels lower than wild-type ExbD. ExbD G96C, K108C, and Y112C also had significant levels of disulfide-linked dimers present. Dimers observed here for F103C were only detected when this substitution was overexpressed. ExbD G96C, with ˜⅔ present as homodimer and ⅓ present as monomer, supported 85% activity, also suggesting that the dimeric form was active. Overexpressed ExbD K108C and Y112C, with dimer levels equal to or greater than monomers, supported 110% and 90% activity, respectively. When levels were decreased to native ExbD levels, significantly less dimer was present for K108C and activity was the same, near 110%. Y112C now showed equal dimer and monomer levels and activity decreased to 80%. The activity of these substitutions could be attributed to the significant level of monomer present.

ExbD homodimers differentially affect ExbD activity. To increase the ratio of dimer to monomer so that the activity of the dimer could be estimated, strains expressing cys substituted ExbDs were treated with the oxidizing agent copper-(1,10-phenanthroline)₃ (CuoP) as described in the Experimental Procedures section below. After treatment, 9 additional cys substitutions formed homodimers. Thus only 4 substitutions, K97C, T114C, K117C, and T121C, still showed no homodimer formation. For E95C, G96C, D99C, and 1102C, homodimer levels remained unchanged after CuoP treatment. Significant increases in homodimers were observed for T109C through E113C, L115C and M116C.

The degree of dimer formation could only be gauged by the decrease in monomer levels. This was due to the fact that, although treated and untreated samples came from the same culture, the detectability of ExbD in the dimer band had greatly increased—exemplified by D111C, Y112C and E113C—suggesting a change in conformation that allowed greater access to antibody binding. Only a slight increase in total ExbD levels was observed when CuoP-treated samples of these cys substitutions were reduced after treatment. Inducer was absent during the 5 min CuoP treatment, ruling out an actual increase in protein synthesized, which would otherwise have been evident in all treated samples.

Since CuoP treatment catalyzed almost 100% dimer formation for some of the cys substitutions, attempts were made to determine activity of the dimeric form. It was necessary to adapt standard CuoP treatments to the demands of the iron transport assays because the standard treatment inhibited iron transport in wild-type cells. The amount of CuoP was reduced 10-fold to 0.03 mM and cells had to be suspended in fully supplemented M9 medium as described in Experimental Procedures.

This treatment no longer catalyzed high levels of dimer formation for the majority of ExbD cys substitutions. Only Y112C and M116C exhibited nearly 100% homodimer. K108C also showed a significant increase in homodimers, though monomer was still present.

For both Y112C and M116C, the dimeric form inhibited iron transport activity. CuoP-treated ExbD Y112C and M116C supported only 5% and 20% activity, respectively, compared to about 90% for buffer only treated samples. In contrast, ExbD K108C exhibited similar monomer levels compared to ExbD M116C after CuoP treatment but was still at least 70% active.

ExbD dimers are assembled with ExbB. It was not known if the inactive ExbD disulfide-linked dimers were inhibited in their ability to assemble as part of a normal complex with ExbB or blocked in later conformational changes after assembly. Both active and inactive ExbD can be crosslinked with formaldehyde to ExbB. Since CuoP catalyzed almost complete dimerization for certain ExbD cys substitutions, attempts were made to formaldehyde crosslink these disulfide-linked dimers to ExbB.

Cultures expressing 4 substitutions that showed the highest dimer levels (K108C, Y112C, E113C, and M116C), were treated with 0.3 mM CuoP 5 min and then crosslinked with formaldehyde under non-reducing conditions. Crosslinked samples were divided in half. One half was reduced with β-mercaptoethanol to hydrolyze disulfide crosslinks, and the other half kept non-reduced. All were solubilized at 60° C. to retain formaldehyde crosslinks. Samples were resolved on reducing or non-reducing SDS-polyacrylamide gels, respectively, and immunoblotted with ExbD-specific polyclonal antibodies. An ExbD disulfide-linked homodimer (˜31 kDa) formaldehyde crosslinked to ExbB (˜26 kDa) would theoretically migrate at approximately 57 kDa. Due to the similar migration to a heterodimeric complex of ExbD and TonB (52 kDa), each substitution was also crosslinked in a ΔtonB background (KP1509) to rule out those complexes due to TonB-ExbD crosslinks.

As seen previously, wild-type monomeric ExbD formaldehyde crosslinked into homodimers and heterodimeric complexes with ExbB and TonB under both non-reducing and reducing conditions. The formaldehyde crosslinked homodimer is typically the complex of lowest abundance but is apparent on long exposures. Under non-reducing conditions, crosslinking profiles for ExbD cys substitutions had much lower levels of the ExbD-ExbB complex and appeared to show a TonB-ExbD crosslink. However, the putative TonB-ExbD bands were still present in a ΔtonB strain, ruling out a crosslink to TonB. The size of the complex suggested it could represent a novel complex of an ExbD disulfide-linked homodimer that was formaldehyde crosslinked to ExbB. Due to the CuoP treatment, much higher levels of homodimers and no monomers were detected. It was not clear why ExbD homodimer bands appeared as a doublet; however, both bands decreased in the presence of reducing agent, suggesting disulfide-linked dimers were in both bands. Doublet bands were also apparent for the ExbD monomer. These may represent two conformations of ExbD or a degradation product.

The same samples were also immunoblotted under reducing conditions that hydrolyzed the disulfide bond of the ExbD homodimer, and retained ExbD monomers formaldehyde crosslinked to other proteins. Under these conditions, levels of suspected ExbD2-ExbB heterotrimer were greatly lowered with concomitant restoration of the typical ExbD-ExbB heterodimer and ExbD monomer band. This confirmed the identity of this band as an ExbD2-ExbB heterotrimer, indicating that ExbD is at least a dimer in complex with ExbB. This also confirmed the supposition, based on data from the Mot and Tol systems, that dimeric ExbD interacts with ExbB. The absence of TonB had no effect on the crosslinking profiles in the reduced samples, indicating that while ExbD was trapped as a disulfide-linked homodimer, it could not formaldehyde crosslink to TonB.

ExbD homodimer formation does not require ExbB or ExbD D25. While it was observed that select ExbD cys substitutions could still complex with ExbB, it was not known if the disulfide-linked dimers formed before or after interaction with ExbB. Since formaldehyde crosslinked ExbD homodimers do not require ExbB, it was possible disulfide-linked dimer formation occurred before interaction with ExbB. The ability of each cys substitution to form spontaneous disulfide-linked dimers was compared in a ΔexbBD, ΔtolQRA strain (RA1017). All dimers still formed and no new dimers were detected in the absence of ExbB. Asp 25 is an essential residue in the ExbD transmembrane domain and a D25N mutation prevents the energized interaction between TonB and ExbD periplasmic domains (Ollis et al., 2009, Molecular microbiology 73: 466-481). A functional ExbD TMD, however, was not important for homodimer formation. Inactive ExbD D25N can be crosslinked into homodimers with formaldehyde, and accordingly, all ExbD D25N cys substitutions still formed disulfide-linked dimers. No new dimers were detected in the presence of the D25N mutation.

ExbD homodimers increase in the absence of TonB. Results above, where formaldehyde crosslinking of disulfide-linked ExbD dimers was analyzed, suggested that the ExbD periplasmic domain could form disulfide-linked homodimers or formaldehyde crosslinked TonB-ExbD heterodimers, but not both at the same time. For the four cys substitutions examined in that assay, dimer formation was efficiently catalyzed even in the absence of TonB. To determine if TonB was important for spontaneous ExbD homodimer formation, disulfide-linked dimer formation was examined in a ΔexbD, ΔtolQR, ΔtonB strain (KP1509). A1114 dimer-forming cys substitutions showed increased disulfide-linked dimers in the absence of TonB.

ExbD cys substitutions that can dimerize also form heterodimers with cys-substituted TonB. It was previously observed that ExbD A92C forms a disulfide-linked heterodimer with TonB(C18G, A150C) (Ollis et al., 2009, Molecular Microbiology 73: 466-481). A92C also forms a low level of disulfide-linked homodimer. To determine if any of the other ExbD cys substituted residues could be trapped in interaction with TonB, each was co-expressed with TonB(C18G, A150C), such that both proteins were equal to native ExbD or TonB levels when protein samples were immunoblotted on reducing SDS-polyacrylamide gels. Reducing and non-reducing samples were immunoblotted with ExbD-specific polyclonal or TonB-specific monoclonal antibodies. A number of ExbD cys substitutions were trapped in disulfide-linked heterodimers with TonB. There was a strong correspondence between those cys substitutions that participated in ExbD-TonB heterodimer formation and those that formed homodimers: A92C, E95C through D99C, K108C, T109C, Y112C, and E113C. ExbD I102C and V110C formed homodimers but not ExbD-TonB heterodimers. K97C and K98C formed heterodimers but not homodimers. Under the conditions of these assays, TonB A150C disulfide-linked homodimers were also observed, with variations in levels corresponding to over- or under-expression of TonB.

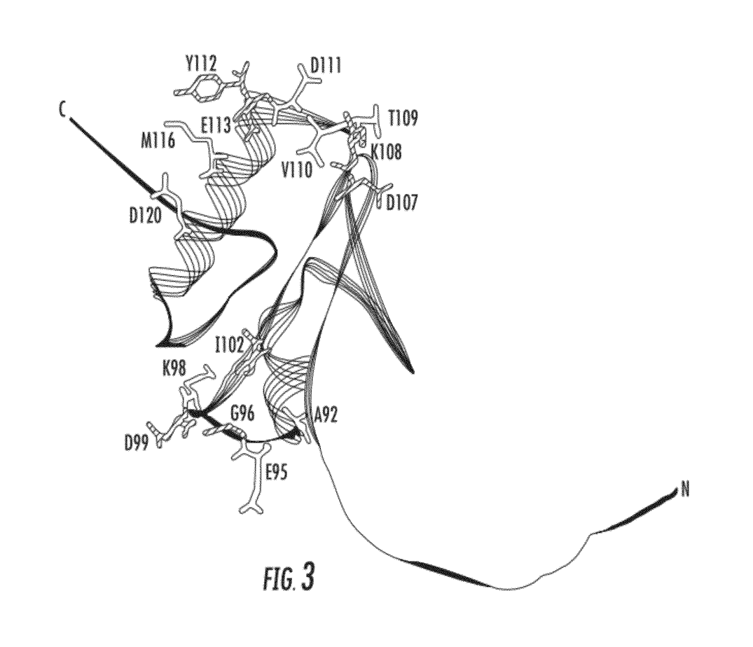

When all sites of spontaneous in vivo disulfide-linked dimer formation in this study were mapped onto the ExbD periplasmic domain NMR structure, homo-dimerization sites clustered at both ends of the structure (FIG. 3). This could suggest the presence of two homodimeric interfaces in vivo. However, no clear interfaces could be identified on the NMR structure, as residue side chains pointed in multiple directions and not all were surface exposed. For example, ExbD I102, which was unstable when substituted with cys unless trapped as a dimer, is a buried residue on the Δ4 strand (FIG. 3), making it difficult to envision how homodimer formation could occur. ExbD I102C was fully active as a dimer, suggesting that this region of the Δ4 strand is surface exposed in the monomers prior to forming the homodimer. Thus the monomeric structure cannot be modeled as a dimer without large conformational distortions.

### Experimental Procedures

Bacterial strains and plasmids. Bacterial strains and plasmid used in this study are listed in Table 5. Single amino acid substitutions were generated for all plasmids using 30-cycle extra-long PCR. All ExbD cys substitutions were constructed using this method with pKP999 (exbD) as the template unless otherwise noted. pKP885, pKP899, pKP905, and pKP911 were derivatives of pKP660 (exbB, exbD). All ExbD D25N cys substitutions were derivatives of pKP1064 (exbD D25N) except for the following: pKP1050 was a derivative of pKP1005 (exbD K97C). pKP1051 was a derivative of pKP1011 (exbD F103C). pKP1052 was a derivative of pKP1017 (exbD T109C). pKP1053 was a derivative of pKP1023 (exbD L115C). pKP1082 was a derivative of pKP1029 (exbD T121C). pKP1429 was a derivative of pKP1024 (exbD M116C). Forward and reverse primers were designed with the desired base change flanked on both sides by 12-15 homologous bases. DpnI digestion was used to remove the template plasmid. Sequences of the exbB segment and exbD gene were confirmed by DNA sequencing.

To construct pKP1005, pKP1011, pKP1017, and pKP1023, forward and reverse primers were designed to amplify the last 22 codons of exbB through the stop codon of exbD from a pKP885 (exbB, exbD K97C), pKP899 (exbB, exbD F103C), pKP905 (exbB, exbD T109C), or pKP911 (exbB, exbD L115C) template respectively, introducing flanking NcoI sites. The PCR-amplified, NcoI-digested fragment was cloned into the unique NcoI site in pPro24. Proper orientation was determined by FspI digestion. Sequences of the exbB segment and exbD gene were confirmed by DNA sequencing.

Media and culture conditions. Luria-Bertani (LB), tryptone (T), and M9 minimal salts were prepared. Liquid cultures and agar plates were supplemented with 100 μg ml-1 ampicillin and plasmid-specific levels of sodium propionate, pH 8, as needed for expression of ExbD. When coexpression of plasmid-encoded TonB (C18G, A150C) was examined, cultures and agar plates were also supplemented with 34 μg ml⁻¹ chloramphenicol and plasmid-specific levels of L-arabinose as needed for TonB expression. M9 salts were supplemented with 0.5% glycerol, 0.4 μg ml⁻¹ thiamine, 1 mM MgSO₄, 0.5 mM CaCl₂, 0.2% casamino acids, 40 μg ml-1 tryptophan, and 37 μM FeCl₃·6H₂O. Cultures were grown with aeration at 37° C. All assays were performed using mid-exponential phase cells (A550≈0.43-0.5, as measured on a spectronic 20 with a path length of 1.5 cm).

Activity assays. Initial rates of [⁵⁵Fe]-ferrichrome uptake were determined. For assays where cultures were treated with copper-(1,10-phenanthroline)₃, harvested cells were pelleted, resuspended in 1×M9 supplemented as described above (no sodium propionate added), and treated with an equal volume 0.06 mM CuoP ([final] 0.03 mM) or 0.5 mM sodium phosphate buffer, pH 7.4 (buffer only) for 5 min, at 37° C. with aeration. Cells were pelleted, and the standard iron transport protocol was followed starting with resuspension in the assay medium.

In initial assays testing CuoP treatment, it was found that resuspension of harvested cells in unsupplemented 1×M9 and subsequent treatment with 0.03 mM CuoP inhibited iron transport of a wild-type strain to about 25%. However, treatment with the same concentration, 0.03 mM CuoP, in supplemented 1×M9 did not inhibit activity of a wild-type strain. In all assays the CuoP solution was removed and assays carried out in identical media, but the medium present during the 5 min CuoP treatment was important and differentially affected activity of a wild-type strain.

In vivo disulfide crosslinking. Saturated LB overnight cultures were subcultured 1:100 in T broth, and assays for spontaneous disulfide crosslinking were performed. Samples were resolved on non-reducing 13% or 11% SDS-polyacrylamide gels and immunoblotted with ExbD-specific polyclonal antibodies or TonB-specific monoclonal antibodies. For reasons that were unclear, anti-ExbD immunoblots from non-reducing gels consistently showed lower detection compared to reducing gels. Typically a 10-minute exposure from a non-reducing gel immunoblot was comparable to intensity of a 1-minute exposure from a reducing gel immunoblot.

For assays catalyzing oxidative crosslinking with copper-(1,10-phenanthroline)₃, saturated LB overnight cultures were subcultured 1:100 in M9 minimal media. Harvested cells were resuspended in unsupplemented 1×M9 and treated with an equal volume 0.06 mM CuoP ([final] 0.03 mM) or 0.5 mM sodium phosphate buffer, pH 7.4 (buffer only) for 5 min, at 37° C. with aeration. Following treatment, samples were precipitated by addition of an equal volume of 20% trichloroacetic acid (TCA). All cell pellets were resuspended in non-reducing Laemmli sample buffer containing 50 mM iodoacetamide. At the time of initial harvest, samples from each culture were also TCA precipitated and pellets resuspended in reducing Laemmli sample buffer containing β-mercaptoethanol (βME). These served as the reduced sample controls for total protein expression levels. Equal A550-mL were loaded for all samples. Immunoblotting was performed as described above.

In vivo formaldehyde crosslinking following oxidative crosslinking. Saturated LB overnight cultures were subcultured 1:100 into M9 minimal media supplemented with sodium propionate. At mid-exponential phase, cells were harvested and treated with an equal volume 0.6 mM CuoP ([final] 0.3 mM) for 5 min, at 37° C. with aeration. Cell pellets were washed once with 1×M9 then resuspended in sodium phosphate buffer pH 6.8 and treated with formaldehyde. Half of the samples were resuspended in non-reducing Laemmli sample buffer containing 50 mM iodoacetamide and half in reducing Laemmli sample buffer containing βME. Samples were resolved on 13% non-reducing or reducing, respectively, SDS-polyacrylamide gels and immunoblotted with ExbD-specific polyclonal antibodies.

TABLE 5
Strains and Plasmids Used in this Study

| Strain or | | |
|---|---|---|
| Plasmid | Genotype or Phenotype | Reference |
| Strains | | |
| W3110 | F⁻ IN(rrnD-rrnE)1 | (Hill & Harnish, 1981, |
| | | Proc. Natl. Acad. Sci. USA |
| | | 78: 7069-7072) |
| RA1017 | W3110, ΔexbBD::kan, | (Larsen et al., 2007, Journal |
| | ΔtolQRA | of bacteriology 189: 2825-2833) |
| RA1045 | W3110, ΔexbD, ΔtolQR | (Brinkman & Larsen, 2008, |
| | | Journal of bacteriology |
| | | 190: 421-427) |
| KP1344 | W3110, tonB, P14::BlaM | (Larsen et al., 1999, Mol. |
| | | Microbiol. 31: 1809-1824) |
| KP1509 | W3110, ΔexbD, ΔtolQR, | (Ollis et al., 2009, |
| | ΔtonB::kan | Molecular microbiology 73: |
| | | 466-481) |
| ^{a}Plasmids | | |
| pKP381 | TonB(H20A) | (Larsen et al., 2007, Journal |
| | | of bacteriology 189: 2825-2833) |
| pKP945 | TonB(C18G, A150C) | (Ollis et al., 2009, |
| | | Molecular microbiology 73: |
| | | 466-481) |
| pKP660 | exbB, exbD in pBAD24 | (Ollis et al., 2009, |
| | | Molecular microbiology 73: |
| | | 466-481) |
| pKP885 | ExbB, ExbD(K97C) | Present study |
| pKP899 | ExbB, ExbD(F103C) | Present study |
| pKP905 | ExbB, ExbD(T109C) | Present study |
| pKP911 | ExbB, ExbD(L115C) | Present study |
| pKP1000 | ExbD(A92C) | (Ollis et al., 2009, |
| | | Molecular microbiology 73: |
| | | 466-481) |
| pKP1049 | ExbD(D25N, A92C) | (Ollis et al., 2009, |
| | | Molecular microbiology 73: |
| | | 466-481) |
| pKP999 | exbD in pPro24 | (Ollis et al., 2009, |
| | | Molecular microbiology 73: |
| | | 466-481) |

| | | |
|---|---|---|
| pKP1001 (L93C) | pKP1011 (F103C) | pKP1021 |
| | | (E113C) |
| pKP1002 (T94C) | pKP1012 (F104C) | pKP1022 |
| | | (T114C) |
| pKP1003 (E95C) | pKP1013 (R105C) | pKP1023 |
| | | (L115C) |
| pKP1004 (G96C) | pKP1014 (A106C) | pKP1024 |
| | | (M116C) |
| pKP1005 (K97C) | pKP1015 (D107C) | pKP1025 |
| | | (K117C) |
| pKP1006 (K98C) | pKP1016 (K108C) | pKP1026 |
| | | (V118C) |
| pKP1007 (D99C) | pKP1017 (T109C) | pKP1027 |
| | | (M119C) |
| pKP1008 (T100C) | pKP1018 (V110C) | pKP1028 |
| | | (D120C) |
| pKP1009 (T101C) | pKP1019 (D111C) | pKP1029 |
| | | (T121C) |
| pKP1010 (I102C) | pKP1020 (Y112C) | |
| pKP1064 | ExbD (D25N) | (Ollis et al., 2009, |
| | | Molecular |
| | | microbiology |
| | | 73: 466-481) |
| pKP1217 (D25N, L93C) | pKP1051 (D25N, F103C) | pKP1234 |
| | | (D25N, E113C) |
| pKP1216 (D25N, T94C) | pKP1226 (D25N, F104C) | pkP1274 |
| | | (D25N, T114C) |
| pKP1218 (D25N, E95C) | pKP1227 (D25N, R105C) | pKP1053 |
| | | (D25N, L115C) |
| pKP1219 (D25N, G96C) | pKP1228 (D25N, A106C) | pKP1429 |
| | | (D25N, M116C) |
| pKP1050 (D25N, K97C) | pKP1261 (D25N, D107C) | pKP1290 |
| | | (D25N, K117C) |
| pKP1233 (D25N, K98C) | pKP1270 (D25N, K108C) | pKP1291 |
| | | (D25N, V118C) |
| pKP1203 (D25N, D99C) | pKP1052 (D25N, T109C) | pKP1294 |
| | | (D25N, M119C) |
| pKP1204 (D25N, | pKP1237 (D25N, V110C) | pKP1297 |
| T100C) | | (D25N, D120C) |
| pKP1260 (D25N, | pKP1262 (D25N, D111C) | pKP1082 |
| T101C) | | (D25N, T121C) |
| pKP1189 (D25N, I102C) | pKP1269 (D25N, Y112C) | |

^{a}Plasmids listed below the lines are derivatives of pKP999 and pKP1064, respectively, unless otherwise noted in Experimental Procedures. The ExbD substitutions expressed from each plasmid are listed in parentheses.

### Example 4

### Identification of Functionally Important TonB-ExbD Periplasmic Domain Interactions In Vivo

In Gram negative bacteria, the cytoplasmic membrane protonmotive force energizes active transport of TonB-dependent ligands through outer membrane TonB-gated transporters. In Escherichia coli, cytoplasmic membrane proteins ExbB and ExbD couple the protonmotive force to conformational changes in TonB, which are hypothesized to form the basis of energy transduction through direct contact with the transporters. While the role of ExbB is not well understood, contact between periplasmic domains of TonB and ExbD is required, with conformational response of TonB to presence or absence of protonmotive force being modulated through ExbD. A region (residues 92-121) within the ExbD periplasmic domain was previously identified as important for TonB interaction. Here the specific sites of periplasmic domain interactions between that region and the TonB carboxy terminus were identified by examining 270 combinations of 45 TonB and 6 ExbD individual cysteine substitutions for disulfide-linked heterodimer formation. ExbD residues A92C, K97C, and T109C interacted with multiple TonB substitutions in four regions of the TonB carboxy terminus Two regions were on each side of the TonB residues known to interact with the TonB box of TonB gated transporters, suggesting that ExbD might position TonB for correct interaction at that site. A third region contained a functionally important glycine residue and the fourth region involved a highly conserved predicted amphipathic helix. Three ExbD substitutions, F103C, L115C, and T121C, were non-reactive with any TonB cysteine substitutions. ExbD D25, a candidate to be on a proton translocation pathway, was important to support efficient TonB-ExbD heterodimerization at these specific regions.

Here we mapped specific disulfide-linked interactions between a subdomain of the ExbD periplasmic domain and the extreme carboxy terminus of TonB in vivo. Multiple significant interactions with TonB cys substitutions were observed for 3 of the 6 ExbD cys substitutions examined, with 3 remaining non-reactive. Interactions clustered in 4 important regions of the TonB carboxy terminus. In the presence of an ExbD D25N TMD mutation, all interactions were absent or significantly reduced.

### Materials and Methods

Bacterial strains and plasmids: Bacterial strains and plasmids used in this study are listed in Table 6.

Media and culture conditions: Luria-Bertani (LB) and tryptone (T) broth were prepared. Liquid cultures and agar plates were supplemented with 34 μg ml⁻¹ chloramphenicol and 100 μg ml⁻¹ ampicillin and plasmid-specific levels of sodium propionate and L-arabinose (percent as w/v), as needed for expression of ExbD and TonB proteins from plasmids. Cultures were grown with aeration at 37° C.

In vivo disulfide crosslinking: Saturated LB overnight cultures were subcultured 1:100 in T broth. Equivalent ODmL of mid-exponential phase cultures were harvested and precipitated by addition of an equal volume of 20% trichloroacetic acid (TCA). Cell pellets were solubilized in non-reducing Laemmli sample buffer containing 50 mM iodoacetamide. Samples were resolved on non-reducing 11% and 13% SDS-polyacrylamide gels and immunoblotted with TonB-specific monoclonal antibodies (Larsen et al., J. Bacteriol. 178:1363-1373, 1996) or ExbD-specific polyclonal antibodies (Higgs et al., Mol. Microbiol. 44:271-281, 2002). Disulfide-linked complexes still formed when samples were not TCA precipitated. Mapping classifications of strong or weak interactions were determined by comparison of at least duplicate crosslinkings to the intensity of representative “strong” (ExbD A92C-TonB P164C) and “weak” (ExbD A92C-TonB Q162C) complexes on TonB-specific immunoblots. Representative complexes were arbitrarily chosen after comparison of band intensities across the full set of crosslinkings.

### Results

3 of 6 ExbD Cys Substitutions Interact Significantly with the TonB Carboxy Terminus In Vivo.

Recently, 70 cys substitutions in the extreme TonB carboxy terminus were characterized, completing a set of 90 single substitutions from TonB A150C through TonB Q239C. From those studies, no single residues were found to be essential for TonB activity. This provided a large pool for studies of TonB interactions through in vivo disulfide crosslinking.

A92 initiates a 30-residue region of ExbD, from 92-121, that is especially important in supporting ExbD protein-protein interactions. Individual cys substitutions were previously constructed spanning these ExbD residues, and all 30 ExbD cys substitutions fully supported TonB activity. Previous work showed that ExbD A92C is trapped in a disulfide-linked heterodimer when co-expressed with TonB A150C (Ollis et al., Mol Microbiol 73:466-81, 2009). In addition to ExbD A92C, ExbD E95C through D99C, K108C, T109C, Y112C and E113C also formed significant heterodimers with TonB A150C. Here we extended our study of specific periplasmic ExbD-TonB interactions to a more comprehensive scan of the extreme carboxy terminus of TonB (every other residue from 150-239).

To examine specific TonB-ExbD interactions in vivo, all plasmids expressing TonB cys substitutions (pACYC ori, cam^{R}) in this study were compatible with plasmids expressing the ExbD cys substitutions (pBR322 ori, amp^{R}). The introduced cys in each protein was the only site of potential disulfide crosslinking, as ExbD has no native cysteine residues, and all TonB cys substitutions were constructed on a cys-less TonB (C18G) (Ghosh and Postle, Mol Microbiol 55:276-88, 2005). TonB and exbD expression were under control of the P_{BAD} and P_{prpB} promoters, respectively, allowing control of expression of both TonB and ExbD substitutions at native levels of each respective protein, as assayed under reducing conditions. Iodoacetamide was also present in the sample buffer to alkylate free sulfhydryl groups and prevent disulfide-linkage from occurring after cell lysis.

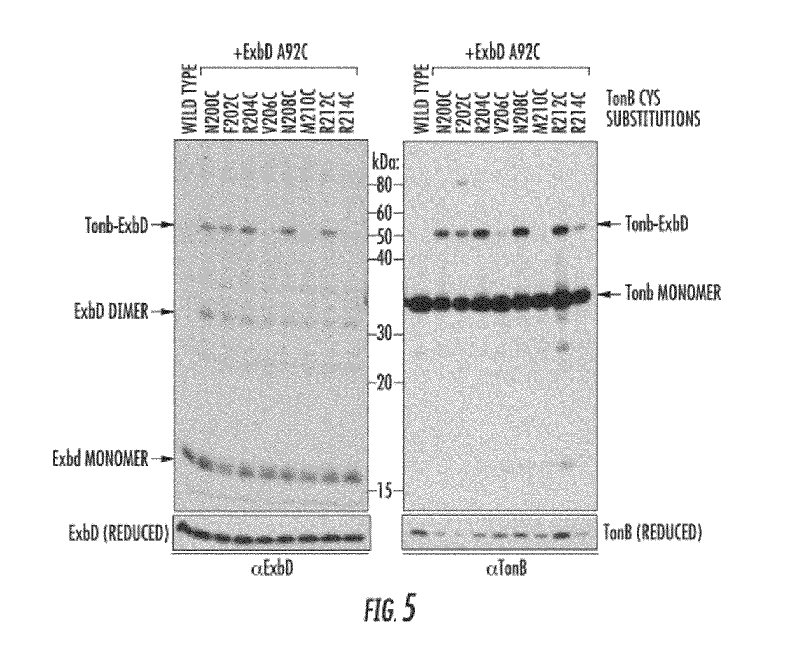

ExbD A92C and every even-numbered TonB cys substitution from A150C to I238C were co-expressed in a ΔexbD, ΔtonB, ΔtolQR strain and analyzed for spontaneous disulfide-linked heterodimer formation on non-reducing SDS-polyacrylamide gels. ExbD- and TonB-specific immunoblots showed the previously observed complex at approximately 52 kDa for ExbD A92C with TonB A150C and significant complexes, designated here as strong interactions, of the same apparent molecular mass for TonB cys substitutions, at 156, 164, 166, 168, 170, 200, 202, 204, 208, and 212. Those interactions are summarized in FIG. 4A, by solid lines. A number of weaker interactions were also observed (FIG. 4A, dashed lines). Any complexes that formed less inefficiently than the “weak” classification were not considered in this study. Immunoblots of ExbD A92C in combination with TonB N200C through R214C are shown as examples of strong (R212C), weak (R214C), and below detection interactions (M210C) (FIG. 5). Subsequent examples show only the anti-TonB immunoblot.

Results with ExbD A92C suggested that specific interactions between TonB and ExbD span the extreme carboxy terminus of TonB. To extend these studies, TonB interactions with ExbD K97C, F103C, T109C, L115C, and T121C, which span the 30-residue ExbD subdomain, were examined. This study, therefore, included 3 previously identified ExbD interactive sites and 3 ExbD sites through which interaction with TonB was not previously detected. Like ExbD A92C, ExbD K97C and T109C exhibited strong and weak interactions with a number of TonB cys substitutions that clustered in similar regions (FIGS. 4B and 4C, solid lines). TonB substitution sites of strong interaction with ExbD K97C were at 150, 170, 184, 202, 204, 208, and 212. Strong TonB interactions with ExbD T109C occurred with TonB residue substitutions at 150, 152, 164, 166, 200, 204, and 208. Three ExbD cys substitutions at F103C, L115C, and T121C were designated as non-reactors because no interactions were detected between them and any of the examined TonB cys substitutions. Degrees of interactions among ExbD A92C, K97C, F103C, T109C, L115C, or T121C with TonB R166C, Q168C, or L170C were determined.

A D25N Mutation in the ExbD TMD Significantly Reduces TonB-ExbD Disulfide-Linked Heterodimer Formation

Since the disulfide-linked heterodimers in this study formed spontaneously, the effect of protonophores on complex formation could not be meaningfully examined. Either the disulfide-linked complexes would be pre-existing in cells when the cys substitutions were expressed at steady state levels, or induction of expression in the presence of protonophores would prevent export of newly synthesized TonB and ExbD cys substitutions from the cytoplasm. Because pmf-dependent interaction between ExbD and TonB periplasmic domains and conformational response of ExbD to the pmf are prevented by a D25N mutation, we used D25N here to mimic the effects of pmf collapse (Ollis et al., Mol Microbiol 73:466-81, 2009). The same 270 TonB-ExbD cys substitution combinations were examined with a D25N mutation in the TMD of all ExbD cys substitutions. Disulfide-linked dimer formation was significantly reduced in the presence of the D25N TMD mutation for all combinations. Of the few heterodimers detected with ExbD D25N cys substitutions, all levels were below those designated as weak interactions, with the caveat that for much darker exposures, some complexes were evident at a low level. The most intense complexes detected in the presence of the D25N mutation were at least 4 times less intense than the strong classification, such as ExbD A92C or T109C crosslinked with TonB P164C. Complexes with D25N were also observed as 2 to 4 times less intense than weak complexes, such as ExbD A92C crosslinked with TonB Q162C or ExbD K97C crosslinked with P164C. For most combinations carrying the D25N substitution, e.g ExbD A92C with TonB Q160C, no complexes were observed. No new heterodimers between the regions of ExbD and TonB examined in this study were detected in the presence of the D25N mutation. Similar to results here with ExbD D25N-TonB interactions, treatment of flagellated cells with the protonophore CCCP prior to BMOE crosslinking through introduced cys substitutions significantly reduces but does not entirely eliminate heterodimeric interactions between ExbD paralogue MotB and flagellar motor protein FlgI compared to when pmf is present.

### Discussion

Recent results from our lab have demonstrated that ExbD forms both homodimers and TonB-ExbD heterodimers in vivo, with nearly the same carboxy terminal periplasmic ExbD residues involved in both interactions. Three stages in the energization of TonB have been identified based on effects of ExbD mutations L132Q in the periplasmic domain and D25N in the transmembrane domain. ExbD L132 is required for an initial correct pmf-independent assembly between TonB and ExbD that subsequently leads to formation of a pmf-dependent conformation detectable by formaldehyde crosslinking through the TonB and ExbD periplasmic domains (Ollis et al., Mol Microbiol 73:466-81, 2009). ExbD D25 is required for the pmf-dependent interaction. Specific residues important in mediating heterodimeric interaction between the TonB and ExbD periplasmic domains, however, were largely unknown.

Here we examined combinations of TonB and ExbD periplasmic domain cys substitutions for spontaneous formation of disulfide-linked heterodimers and the importance of ExbD D25 in complex formation. The targeted regions in this study were the TonB extreme carboxy terminus (residues 150-239), where the only functionally important TonB periplasmic domain residues are located (Postle et al., MBio 1, 2010) and a 30-residue region of the ExbD periplasmic domain (residues 92-121) important in supporting protein-protein interactions of ExbD. A significant number of TonB cys substitutions were trapped in spontaneous disulfide-linked interaction through 3 of the 6 ExbD cys substitutions examined, with the other 3 showing essentially no reactivity. These were designated non-reactors. When all sites of heterodimer formation were compared, 4 regions of ExbD-TonB interactions were apparent, based on TonB sites where at least one ExbD cys substitution exhibited strong interaction or at least 2 exhibited weak interactions.

ExbD Contacts Four Regions of the Extreme TonB Carboxy Terminus

The first and second regions included TonB A150C and G152C, and TonB P164C, R166C, Q168C, and L170C, respectively. Both of these regions are located towards the carboxy terminal end of a large region of TonB periplasmic domain (residues 33-169) that is predicted to be disordered, and residues 103-151 are disordered in vitro (Peacock et al., J Mol Biol 345:1185-97, 2005; Larsen et al., J Bacteriology 189:2825-2833, 2007). The predicted unstructured nature of this region may facilitate interaction of ExbD at multiple sites. Close interaction of ExbD with this unstructured region would support a previously proposed role for ExbD in the potential disorder-to-order transitions of this region of TonB (Vakharia-Rao, et al., J Bacteriol 189:4662-70, 2007).

The first and second regions are also found on either side of a region of TonB, residues 159-164, that makes direct in vivo contact with the conserved amino terminal TonB boxes of the OM TonB-gated transporters BtuB and FecA (Cadieux et al., J Bacteriol 182:5954-61, 2007; Ogierman, M., and V. Braun, J Bacteriol 185:1870-85, 2003). This result, that TonB interacted with ExbD at sites unique but adjacent to interactions with transporter TonB boxes, suggested that ExbD could theoretically interact simultaneously with TonB that is bound to the TonB box or possibly even direct bound TonB to the TonB box. Perhaps ExbD “primes” TonB for proper TonB box interaction by stabilizing a specific conformation of the periplasmic domain, similar to the priming of pilus subunits, for subsequent donor strand exchange, by a periplasmic chaperone in the chaperone-usher pathway.

The third region of ExbD-TonB interaction was TonB P184C and G186C, which exhibited primarily weak interactions with 2 of the 3 reactive ExbD cys substitutions, A92C and K97C. G186 is one of the only 7 residues, and the only non-aromatic residue, in the TonB periplasmic domain that is functionally important. The high conservation of G186, compared to the other functionally important residues, led to the proposal that it may have a fundamental mechanistic role, as opposed to being a site of direct transporter recognition by TonB, as the other aromatic residues are hypothesized to be. It may be that interaction with this region is important for ExbD to catalyze TonB conformational changes.

The fourth region was characterized by strong interaction of the three reactive ExbD cys substitutions with TonB N200C, F202C, R204C, N208C, and R212C. This region is found within a predicted amphipathic helix (residues 199-216) of TonB, one of the most highly conserved TonB features across Gram negative bacteria, though its functional significance is unknown. Of the even-numbered TonB residues exhibiting strong ExbD interactions, N200, R204, N208, and R212 mapped to one face of this helix in the crystal structures of TonB periplasmic domain fragments. The exception was F202, which maps to the opposite face of the helix. In the monomeric TonB NMR structure (Peacock et al., Biometals 19:127-42, 2006) or crystal structures of TonB in complex with BtuB or FhuA (Pawelek et al., Science 312:1399-402, 2006; Shultis et al., Science 312:1396-9, 2006). N200 and F202 are not part of the helix but in an adjacent loop. The alignment of strong interactive sites almost exclusively to one face of the predicted helix suggests that it is partially solvent exposed at some point in the energy transduction cycle. Strong interactions between ExbD A92C or K97C with these TonB residues on one face of this helix and with TonB F202C, on the opposite face, suggested ExbD interacted with multiple conformations of TonB. F202 is a buried residue in both dimeric TonB structures, inaccessible to interaction with ExbD, suggesting this helix may be a dynamic structural element.

A Model for the Role of Pmf in ExbD-TonB Interactions

The overall lack of ExbD D25N interaction with the TonB extreme carboxy terminus (residues 150-239) provided a rationale for the proteinase K resistance of the amino-terminal 1 to ˜156 residues of TonB and concomitant proteinase K sensitivity of residues ˜157-239 that occurs in the presence of ExbD D25N. Perhaps the pmf independent interaction between TonB and ExbD involves TonB residues 1-156, with contact expanding to include the entire TonB carboxy terminus as pmf is utilized. The low level detection of some heterodimers with ExbD D25N cys substitutions suggested D25 was important for either the formation of the complexes, such that they formed inefficiently in the presence of D25N, or for maintaining ExbD-TonB periplasmic domains in the crosslinkable conformations. Strong interactions in the presence of D25 may reflect the conformational changes due to response to pmf.

Approximately 40% of wild-type TonB fractionates with the OM following sucrose density gradient fractionation. Because TonB remains associated with the CM during its energy transduction cycle, this fraction appears to represent TonB that is so tightly bound to the OM that it can be pulled out of its association with ExbB/ExbD during the fractionation process. Formation of TonB triplet homodimers through the extreme carboxy terminus can preclude this interaction of TonB with the OM, with disulfide-linked complexes detected only with CM fractions. It is hypothesized that these TonB homodimeric interfaces must normally be reorganized before the TonB periplasmic domain can interact with OM proteins (Ghosh, J., and K. Postle, Mol Microbiol 55:276-88, 2005). The formation of TonB-ExbD heterodimers through sites common to TonB homodimer formation, such as G186C and F202C, further supported the idea that the TonB interfaces represented by the triplet homodimers are not permanent. Because significant TonB-ExbD interactions in this study required ExbD TMD residue D25, these heterodimeric interactions appeared to occur in response to pmf. These data were consistent with previous observations that a formaldehyde-specific crosslink between TonB and ExbD periplasmic domains requires the pmf. Taken together, these studies extended our data on ExbD-directed remodeling of the TonB periplasmic domain described above to involve a network of specific D25-promoted ExbD-TonB interactions. This work further suggested that one role of these interactions might be to resolve TonB homodimeric interactions to free sites for interaction with OM TonB-gated transporters.

Implications from Solved Structures of ExbD and TonB Carboxy Termini

The solved structures of the TonB carboxy terminus are all similar, with two main conformational variations observed, whether the TonB fragment is monomeric or in complex. The specific sites of TonB-ExbD interactions observed in vivo mapped primarily along one side of these structures. In the TonB₁₆₅₋₂₃₉ homodimer, most sites exhibiting strong interactions with ExbD were exposed. This differs from TonB₁₅₃₋₂₃₉ crystallized in complex with the OM transporter BtuB, where the general region involved in interaction with ExbD interfaced with the periplasmic face of the transporter. TonB residues forming interactions with BtuB in vitro spanned the regions from 158 to 172, 199 to 213, and 225 to 233 [analyzed using the program MONSTER (Salerno et al., Nucleic Acids Res 32:W566-8, 2004)]. TonB R166, L170, N200 and R204, which all exhibited strong interactions with ExbD in vivo, are involved in specific interactions with the β-barrel of BtuB in vitro. R166, Q168, and L170 also exhibit interactions with the plug domain of BtuB. It is unknown if these specific TonB-BtuB interactions occur in vivo, but this might identify some common TonB interactive sites that are shared with both ExbD and TonB-gated transporters. It is known that the TonB box is not the only site through which TonB interacts with the transporters in vivo, though specific sites remain to be determined Two TonB regions, from 158-163 and 226-233, exhibit extensive in vitro interactions with residues of the BtuB TonB box (residues 6-12) in the co-crystal structure. It is notable that neither of these regions exhibited strong in vivo interactions with ExbD. Additionally TonB P184 and G186, exhibited interactions specific to ExbD in vivo. While unique sites of interaction may relate to functions specific to the individual partner proteins, TonB sites common to interaction with both proteins may have mechanistic implications. Sequestering of interaction sites by one partner may be important in the relay of signals or directing a sequence of interactions between the multiple protein partners in the TonB system.

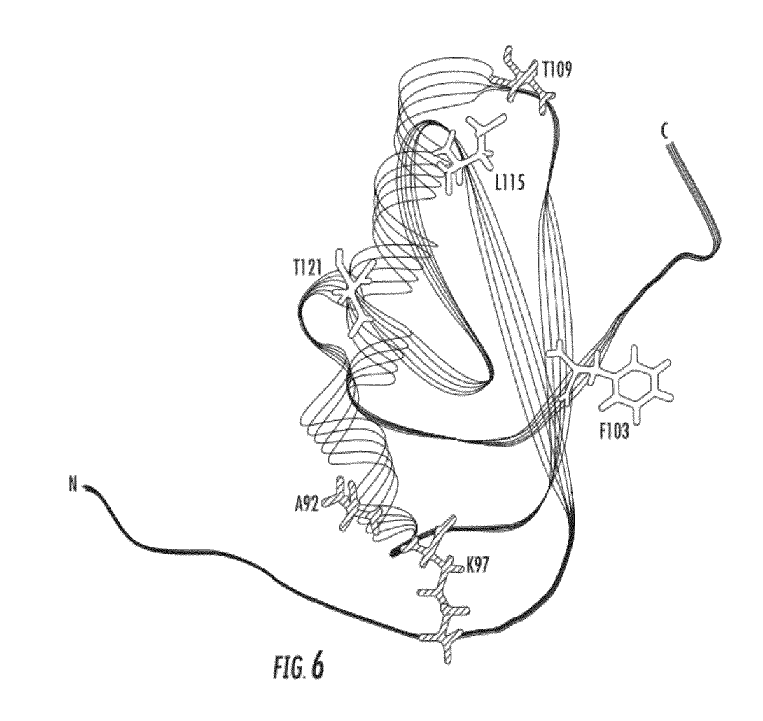

An important finding of this work is that the same three ExbD residues were involved in all interactions with the TonB carboxy terminus, with the remaining three ExbD cys residues exhibiting no interactions. When the three sites of significant ExbD interactions with the TonB carboxy terminus were mapped on the ExbD periplasmic domain NMR structure, A92 and K97 were located at one end of the structure, with T109 at the opposite end, suggesting the possibility of multiple interfaces of interaction of the ExbD periplasmic domain with TonB (FIG. 6). The non-reactors F103, L115, and T121 were each positioned closer to the middle of the structure. Although the periplasmic domains of TonB and ExbD clearly interact in vivo, no significant interactions are observed in vitro, either at pH 3, which allowed solution of the monomeric ExbD NMR structure, or pH 7, which approximates the pH of the periplasm. The TonB carboxy terminal fragment used in those studies (residues 103-239) assumes a nearly identical conformation to the monomeric unit of TonB₁₅₃₋₂₃₉ crystallized in complex with BtuB. Thus these observed conformations do not appear to represent the interactive states of the ExbD and TonB periplasmic domains in vitro or in vivo. Nevertheless, elements of the structures, such as the TonB amphipathic helix, may mediate important interactions for energy transduction in vivo. Localized secondary structural elements could provide interactive surfaces, but perhaps the stable tertiary structures observed in vitro lack the proper context for these elements that would normally allow functional interactions to occur.

This work provided a first extensive view of specific and apparently pmf-promoted interactions between TonB and ExbD periplasmic domains in vivo. By identifying TonB regions involved in interactions with both ExbD and TonB gated transporters, these studies supported a role for ExbD in regulating TonB conformation and suggested a model where ExbD may transfer TonB to specific interactions with the transporters.

TABLE 6
Strains and plasmids used in this study.

| Strain or Plasmid | Genotype or Phenotype |
|---|---|
| Strains | |
| W3110 | F⁻ IN(rrnD-rrnE)1 |
| KP1509 | W3110, ΔexbD, ΔtonB::kan, ΔtolQR |
| ^{a}Plasmids | |
| pKP945 | TonB C18G, A150C |
| pKP1000 | ExbD A92C |
| pKP1005 | ExbD K97C |
| pKP1011 | ExbD F103C |
| pKP1017 | ExbD T109C |
| pKP1023 | ExbD L115C |
| pKP1029 | ExbD T121C |
| pKP1049 | ExbD D25N, A92C |
| pKP1050 | ExbD D25N, K97C |
| pKP1051 | ExbD D25N, F103C |
| pKP1052 | ExbD D25N, T109C |
| pKP1053 | ExbD D25N, L115C |
| pKP1082 | ExbD D25N, T121C |

| | | | |
|---|---|---|---|
| pKP947 (G152C) | pKP597 (G174C) | pKP609 (P198C) | pKP508 |
| | | | (F180C) |
| pKP949 (R154C) | pKP598 (V176C) | pKP643 (G218C) | pKP469 |
| | | | (N200C) |
| pKP951 (L156C) | pKP601 (V178C) | pKP617 (P220C) | pKP415 |
| | | | (F202C) |
| pKP953 (R158C) | pKP604 (V182C) | pKP619 (S222C) | pKP418 |
| | | | (R204C) |
| pKP588 (Q160C) | pKP610 (P184C) | pKP623 (I224C) | pKP463 |
| | | | (V206C) |
| pKP587 (Q162C) | pKP612 (G186C) | pKP625 (V226C) | pKP416 |
| | | | (N208C) |
| pKP585 (P164C) | pKP614 (V188C) | pKP627 (I228C) | pKP466 |
| | | | (M210C) |
| pKP589 (R166C) | pKP638 (N190C) | pKP629 (I232C) | pKP471 |
| | | | (R212C) |
| pKP593 (Q168C) | pKP639 (Q192C) | pKP631 (G234C) | pKP473 |
| | | | (R214C) |
| pKP591 (L170C) | pKP641 (L194C) | pKP634 (T236C) | pKP475 |
| | | | (E216C) |
| pKP600 (I172C) | pKP607 (A196C) | pKP636 (I238C) | pKP510 |
| | | | (F230C) |

^{a}Plasmids listed below the line express TonB C18G with the cys substitution listed in parentheses. Plasmids in the first 3 columns are from Postle, K., K. A. Kastead, M. G. Gresock, J. Ghosh, and C. D. Swayne, The TonB dimeric crystal structures do not exist in vivo. MBio 1, 2010. Plasmids in the last column are from Ghosh, J., and K. Postle, Mol Microbiol 55: 276-88, 2005.

### Example 5

### Development of Novel Antibiotics

A 30-residue region within the periplasmic domain of ExbD likely to interact with TonB residues was identified. ExbD A92C, K97C, and T109C trap functionally relevant disulfide contacts with several TonB residues. The ExbD-TonB interaction will be targeted by use of homologous competing peptides, with sequences based on the interactive regions of ExbD. There may also be other regions to target, since the ExbD L132Q substitution is inactive and prevents formation of the TonB-ExbD formaldehyde crosslink.

Antibiotics to be tested will typically be small stable peptides based on the ExbD (or TonB) periplasmic amino acid sequences. To ensure that the peptides are proteolyticially stable, an intein system (SICLOPPS) will be used to produce a circular peptide from ribosomally synthesized precursors. Inteins are naturally occurring protein splicing sequences that can be used to ligate N and C termini of a peptide to circularize it. Basically, the desired codon sequence is engineered between C-terminal (I_{C}) and N-terminal (I_{N}) intein coding sequences. Recovery of cyclic peptides for characterization and purification is aided by a chitin binding domain (CBD) fused to the C-terminus of I_{N}. Expression of the plasmid encoded fused I_{C}-ExbD codons-I_{N}-CBD peptide is induced, cells are lysed by French press in buffer appropriate for the chitin column and the lysate is allowed to process on the chitin column down to the circularized peptide and various intermediates. Here, target sequences are known and inhibitory peptides can be designed.

Modification of intein residues may be useful to optimize the cyclization reaction. For rapid screening of various constructs, lysates of cells expressing the entire fusion construct are first assayed for their ability to inhibit the formation of proteinase K resistant TonB complexes in spheroplasts, the Stage II interaction between TonB and ExbD periplasmic domains. Promising constructs will then be optimized for cyclization on the chitin affinity column and tested for inhibition of [⁵⁵Fe]-ferrichrome transport.

9-mer, and perhaps larger, circular peptides can cross the E. coli OM. The spheroplast screening will serve to determine whether the circular peptides are too large to cross the OM, if the peptides are active only on spheroplasts. It is hypothesized that bacteria can become resistant to the effects of the peptide by mutation such that the partner protein TonB (or ExbD) no longer interacts with it. However in that case, the resistant mutants will also no longer be able to function in establishing a functional ExbD-TonB interaction. Thus they should be unable to energize high affinity iron siderophore transport across the OM. Bacterial growth is inhibited under either circumstance. To test that hypothesis, resistant mutants (e.g. to active circular peptides corresponding to ExbD sequences) will be isoalated, iron transport will be assayed, and the corresponding interacting partner (TonB) sequenced to confirm the source of the resistance.

As described above, the sites of in vivo interaction between TonB and FepA, including the FepA TonB box, were defined. These various FepA sequences will be used as templates for circular peptide engineering. Such sites are expected to vary more among Gram negative bacteria than sites of ExbD-TonB interaction, however they may also prove to be valuable targets Inhibition by purified circular peptides will be identified as a decrease in colicin B or phage H8 killing. Based on the similarity of FepA and FhuA sequences and crystal structures, equivalent sites will also be engineered using FhuA sequences where a decrease in ferrichrome transport can be monitored.

As with the TonB system, TolR-TolA interactions will be targeted. The Tol system is sufficiently similar to the TonB system that there is functional crosstalk between them (Eick-Helmerich and Braun, J. Bacteriol. 1989 September; 171(9):5117-26; Braun and Hermann, Mol. Microbiol. 1993 April; 8(2):261-8). ExbD and TolR have similarity in their sequences. The Tol system is involved in maintenance of OM integrity (Cascales et al., Microbiol Mol Biol Rev. 2007 March; 71(1):158-229). If it could be targeted by a cyclic peptide, it could prove an important adjunct to many different antibiotics, including those targeting the TonB system, by making the OM leaky. The same strategy as above will be employed, using the regions of TolR most likely to interact with TolA as templates for engineered circular peptides. The test of the peptides will be whether they can decrease the minimal inhibitory concentration of antibiotics, such as vancomycin, which are commonly used to assay OM leakiness induced in Tol system mutants (Cascales et al., Microbiol Mol Biol Rev. 2007 March; 71(1):158-229). Should such peptides be identified, it will be determined if it is possible to engineer larger (and thus potentially more potent) ExbD-specific circular peptides.

Inhibitory circular peptides on Acinetobacter baumanii will be tested. A. baumanii is a globally emerging Gram negative pathogen that rapidly acquires antibiotic resistance in hospital settings. It is the cause of pneumonias, meningitis, septicemia, and urinary tract infections and has recently been the causative agent of severe infections in military personnel wounded in Iraq and Afganistan. The tonB, exbB, exbD operon of A. baumanii 17978 complements a ΔexbB,exbD strain of E. coli K12. It could not complement a ΔtonB strain, possibly because A. baumanii TonB interacts with a hemin-specific TGT not found in E. coli. (Zimbler et al., Biometals. 2009 February; 22(1):23-32). It is hypothesized that circular peptides corresponding to E. coli ExbD sequences will also be inhibitory to A. baumanii growth on iron limiting medium. The applicability of circular TolR peptides on increasing sensitivity to antibiotics will be tested.

### Other Embodiments

Any improvement may be made in part or all of the compositions and method steps. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. Although the experimental data described herein pertain to the TonB system, targeting TolR-TolA interactions in the Tol system is also encompassed, and may be used to develop compositions for inhibiting growth of gram-negative bacteria in a subject. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contraindicated by context.

What is claimed is:

1. A pharmaceutical composition comprising an inhibitor of a TonB/ExbD interaction in a therapeutically effective amount for inhibiting growth of gram-negative bacteria in a subject and a pharmaceutically acceptable carrier.

2. The pharmaceutical composition of claim 1, wherein the inhibitor is a peptide.

3. The pharmaceutical composition of claim 2, wherein the peptide is a cyclic peptide of approximately 9 or greater amino acids, is produced by Split Intein Circular Ligation of Proteins and Peptides (SICLOPPS) methodology, and is an antibiotic.

4. The pharmaceutical composition of claim 1, wherein the inhibitor is a modified colicin protein and an antibiotic.

5. The pharmaceutical composition of claim 1, wherein the gram-negative bacteria are selected from the group consisting of: Escherichia coli, Salmonella enterica serovar typhi, Vibrio cholerae, Burkholderia pseudomallei, Klebsiella pneumoniae, Pseudomonas aeruginosa, Acinetobacter baumanii, and Yersinia enterocolitica.

6. The pharmaceutical composition of claim 1, wherein the inhibitor decreases uptake of iron by the gram-negative bacteria.

7. The pharmaceutical composition of claim 1, wherein the inhibitor binds to a region of ExbD comprising amino acids 42-61, 62-141, or 92-121 of ExbD, or to a region of TonB comprising amino acids 33-239 of TonB.

8. A method of inhibiting growth of gram-negative bacteria in a subject, the method comprising the steps of:
a) providing a pharmaceutical composition comprising an inhibitor of a TonB/ExbD interaction in a therapeutically effective amount for inhibiting growth of gram-negative bacteria in the subject and a pharmaceutically acceptable carrier; and
b) administering the pharmaceutical composition to the subject.

9. The method of claim 8, wherein the subject is a human, and the inhibitor enters the periplasmic space of the gram-negative bacteria and inhibits their growth.

10. The method of claim 8, wherein the inhibitor is a peptide.

11. The method of claim 10, wherein the peptide is a 9-mer or larger cyclic peptide produced by SICLOPPS methodology and is an antibiotic.

12. The method of claim 8, wherein the inhibitor is a modified colicin protein and is an antibiotic.

13. The method of claim 8, wherein the gram-negative bacteria are selected from the group consisting of but not limited to: Escherichia coli, Salmonella enterica serovar typhi, Vibrio cholerae, Burkholderia pseudomallei, Klebsiella pneumoniae, Pseudomonas aeruginosa, Acinetobacter baumanii, and Yersinia enterocolitica.

14. The method of claim 8, wherein the inhibitor decreases uptake of iron by the gram-negative bacteria.

15. The method of claim 8, wherein the inhibitor binds to a region of ExbD comprising amino acids 42-61, 62-141, or 92-121 of ExbD, or to a region of TonB comprising amino acids 33-239 of TonB.

16. A method of inhibiting growth of gram-negative bacteria on a solid surface, the method comprising:
a) providing a composition comprising an inhibitor of a TonB/ExbD interaction in a therapeutically effective amount for inhibiting growth of gram-negative bacteria; and
b) coating the solid surface with the composition in an amount effective for inhibiting growth of the gram-negative bacteria on the solid surface.

17. The method of claim 16, wherein the solid surface is at least one surface of a medical device or any other solid surface subject to biofouling, and the inhibitor enters the periplasmic space of the gram-negative bacteria and inhibits their growth.

18. The method of claim 16, wherein the inhibitor is a 9-mer or larger cyclic peptide produced by SICLOPPS methodology and is an antibiotic.

19. The method of claim 16, wherein the inhibitor is a modified colicin protein and is an antibiotic.

20. The method of claim 16, wherein the gram-negative bacteria are selected from the group consisting of: Escherichia coli, Salmonella enterica serovar typhi, Vibrio cholerae, Burkholderia pseudomallei, Klebsiella pneumoniae, Pseudomonas aeruginosa, Acinetobacter baumanii, and Yersinia enterocolitica.

21. The method of claim 16, wherein the inhibitor reduces uptake of iron by the gram-negative bacteria.

22. The method of claim 16, wherein the inhibitor binds to a region of ExbD comprising amino acids 42-61, 62-141, or 92-121 of ExbD, or to a region of TonB comprising amino acids 33-239 of TonB.
